(19) United States
(12) Patent Application Publication
(10) Pub. No.: US 2023/0112104 A1
(43) Pub. Date: Apr. 13, 2023

(54) METHODS AND SYSTEMS FOR CELL SEPARATION

(71) Applicant: LevitasBio, Inc., Menlo Park, CA (US)

(72) Inventors: Kathy Lazaruk, Redwood City, CA (US); Christopher Stevens, Fremont, CA (US); Kevin Travers, Menlo Park, CA (US)

(73) Assignee: LevitasBio, Inc., Menlo Park, CA (US)

(21) Appl. No.: 18/045,472

(22) Filed: Oct. 11, 2022

Related U.S. Application Data
(60) Provisional application No. 63/362,630, filed on Apr. 7, 2022.
(60) Provisional application No. 63/254,944, filed on Oct. 12, 2021.

(51) Int. Cl. B01L 3/00; G01N 15/10

(52) U.S. Cl. CPC B01L 3/502761; G01N 15/1031; B01L 2200/0652; B01L 2200/04; B01L 2300/0883; B01L 2300/0864; G01N 2015/1081

(57) ABSTRACT

Described are various embodiments of methods, devices, systems and kits for magnetic levitation-based separation of mixtures or populations of particles that include various types of particles. Some embodiments of such methods, devices, systems and kits are useful for magnetic levitation-based separation of mixtures or populations of cells that include various cell types. Some other embodiments of the described methods, devices, systems and kits are useful for magnetic levitation-based separation of mixtures or population of cellular or mixtures or population of biological molecules.

### CROSS-REFERENCES TO RELATED APPLICATIONS

This application claims the benefit of U.S. Provisional Application No. 63/362,630, filed Apr. 7, 2022, and U.S. Provisional Application 63/254,944 filed Oct. 12, 2021. The entire contents and disclosures of these applications are incorporated herein by reference.

### BACKGROUND OF THE INVENTION

Magnetic levitation recently emerged as a useful method for separating particles, including cells and biological molecules. During magnetic levitation a particle suspended in a paramagnetic fluid medium is exposed to a magnetic field gradient, which generates a non-uniform pressure equivalent to the magnetic energy density in the paramagnetic fluid medium. In a magnetic field gradient, the particles (or “objects”) subjected to magnetic levitation appear to be repelled from the regions of high magnetic field. In actuality, the object is displaced by an equal volume of the paramagnetic fluid medium. The attractive interaction between the paramagnetic fluid medium and the regions of high magnetic field results in magnetic levitation of the object. Conditions and systems have been established for levitating live cells, and it has been demonstrated that both eukaryotic and prokaryotic cells have unique magnetic levitation profiles. See Durmus et al., 2015, “Magnetic levitation of single cells,” Proc Natl Acad Sci USA 112(28):E3661-8 (“Durmus”). Systems for collecting cells at different levitation heights and collecting them for downstream analysis and other applications have been developed.

### BRIEF SUMMARY OF THE INVENTION

The terms “invention,” “the invention,” “this invention” and “the present invention,” as used in this document, are intended to refer broadly to all of the subject matter of this patent application and the claims below. Statements containing these terms should be understood not to limit the subject matter described in the present disclosure or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention and described in the present disclosure are methods of cell separation, comprising the steps of: binding a first density modifying agent to a cell of a first type in a population of cells comprising multiple cell types, wherein the first density modifying agent comprises a first non-magnetic microparticle and a first linking agent that preferentially binds to cells of the first type, thereby forming a first complex, said first complex comprising the first density modifying agent bound to an individual cell of the first type; forming a suspension in a paramagnetic fluid medium, the suspension comprising a plurality of the first complexes and a plurality of the cells of the multiple cell types; introducing the suspension into a processing channel of a flowcell cartridge; and exposing the processing channel to a magnetic field for a first period of time sufficient for at least some of the first complexes to separate in the processing channel from the cells of the multiple cell types not bound by the first density modifying agent, thereby forming a first portion of the suspension, wherein the first portion is enriched with the first complex relative to the suspension, and a second portion of the suspension, the second portion depleted in the first complex relative to the suspension. Also included among the embodiments of the present invention and described in the present disclosure are magnetic levitation kits comprising a paramagnetic fluid medium and one or more of density modifying agents or separate components of the one or more density modifying agents, capable of forming complexes with individual cells, wherein density of each of the complexes is different than density of the individual cells, and wherein each density modifying agent comprises a non-magnetic microparticle and a linking agent that preferentially binds to a target cell type. Also included among the embodiments of the present invention and described in the present disclosure are systems for cell separation, comprising: a first non-magnetic microparticle capable, alone or in combination with first other reagents, of preferentially binding to cells of a first type in a population of cells comprising multiple cell types and forming a first complex of the microparticle and a cell of the first type, the first complex having density that is different from density of the cell of the first type and from other types of cells of the multiple cell types; a flowcell cartridge comprising a first outlet channel and a processing channel; a station comprising a holding block for the flowcell cartridge and one or more magnets positioned to expose the processing channel of the flowcell cartridge located in the holding block to a magnetic field, wherein exposing to the magnetic field the processing channel of the flowcell cartridge containing a suspension of the cells of the multiple cell types in a paramagnetic fluid medium allows the first complex to separate in the processing channel from the cells of the multiple cell types not bound by the first non-magnetic microparticle and from the other types of cells of the multiple cell types.

### BRIEF DESCRIPTION OF THE DRAWINGS

FIG. 1 is a schematic representation of an exemplary magnetic levitation system.

FIG. 2 is a schematic representation of two views of an exemplary flowcell cartridge of a magnetic levitation system.

FIG. 3 is a schematic representation of a method according to an exemplary embodiment of the present invention.

FIG. 4 is a schematic representation of a method according to an exemplary embodiment of the present invention.

FIG. 5 is a schematic representation of a cell complexed with a microbubble according to some embodiments described in the present disclosure.

FIG. 6 are photographic images illustrating separation of cells complexed to microbubbles according to some embodiments described the present disclosure.

FIG. 7 is a box plot illustrating separation of cells complexed to microbubbles according to some embodiments described the present disclosure.

FIG. 8 is a schematic representation of a cell complexed with a microbead according to some embodiments of the present disclosure.

FIG. 9 is a photographic image illustrating separation of cells complexed to polymeric microbeads according to some embodiments described the present disclosure.

FIG. 10 are photographic images illustrating separation of cells complexed to gold microbeads according to some embodiments described the present disclosure.

FIG. 11 is a dot plot showing the levitation height of cells in the absence of an antibody coupled to a gold nanoparticle, and in the presence of an antibody coupled to a gold nanoparticle.

FIG. 12 is a schematic illustration of the model of the microparticles complexed to cell surface.

FIG. 13 is a table illustrating density calculation for microparticle-cell complexes, with the microparticles having a density of 1.063 g/cm³, and the cell having a diameter of 11.5 μm.

### DETAILED DESCRIPTION OF THE INVENTION

Cells have inherent properties that dictate their behavior when subjected to magnetic levitation. Durmus et al. showed that the height at which a cell levitates in a paramagnetic fluid medium (“levitation profile”) corresponds to cellular density, and that different cell types can be distinguished based on their characteristic magnetic levitation profiles. See also co-pending, commonly owned U.S. patent application Ser. No. 17/449,438, filed Sep. 29, 2021, incorporated by reference herein, which describes methods and systems for separation of cells by magnetic levitation, including the methods for separation of dead cells from living cells. Described in the present disclosure are improved processes (methods), devices, systems and kits for separation of particles, including cells, by magnetic levitation.

The inventors discovered methods that improve separation of particles, such as cells, during a magnetic levitation process. The inventors found that, by binding cells to non-magnetic microparticles, they were able to alter the levitation height of the cells, when the cells were suspended in a paramagnetic fluid medium in a flowcell cartridge of a magnetic levitation system and exposed to a magnetic field. Levitation height may be defined by vertical position of the cells in a flowcell cartridge of a magnetic levitation system. In other words, the inventors were able to control the levitation height of the cells during magnetic levitation process by complexing the cells with density-modifying non-magnetic microparticles. In one example, cells with cell-specific surface markers were linked to low-density non-magnetic microparticles coupled to anti-surface marker antibodies. The resulting complexes were suspended in a paramagnetic medium, and magnetically levitated in a processing channel of a flowcell cartridge of a magnetic levitation system. Provided that the density of the cells bound to the non-magnetic microparticles (cell-microparticle complexes) was lower than that of the unbound (uncomplexed) cells, complexes of cells with the low-density non-magnetic microparticles levitated higher in the processing channel than the same cells not complexed to the low-density non-magnetic microparticles. In another example, cells with cell-specific surface markers were complexed with high-density non-magnetic microparticles coupled to anti-surface marker antibodies, the resulting complexes were suspended in a paramagnetic medium, and levitated in a processing channel of a flowcell cartridge of a magnetic levitation system. Provided that the density of the cells bound to the non-magnetic microparticles (cell-microparticle complexes) was higher than that of the unbound (uncomplexed) cells, complexes of cells with the high-density non-magnetic microparticles levitated lower in the processing channel than the same cells not complexed to the high-density microparticles. By coupling density-modifying non-magnetic microparticles to antibodies specific to surface markers characteristic of a cell type of interest found in a population of cells containing multiple cell types, the inventors were able to selectively alter levitation height of the cells belonging to the cell type of interest.

In the above examples, the levitation height of the complexes of cells with microparticles is affected by the number of the microparticles in each complex. The number of microparticles per cell in the complexes can be varied by changing the ratio of particles to cells during complex formation (“PTC ratio”) from about 1 to about 100,000. PTC ratio can also be referred to as particle-to-cell ratio, microparticle-to-cell ratio, bead-to-cell ratio, or by other related terms and expressions. The inventors found that increasing the PTC ratio lowered the levitation height of the complexes. In some cases, it may be beneficial to use smaller microparticles in order to achieve higher concentration during complex formation, which may result in increased PTC ratios. The inventors also found that various other parameters, in addition to the PTC ratio and the applied magnetic field strength, affected the levitation height of the complexes the cells with microparticles. Some of these parameters are the properties of materials included in the microparticle, microparticle size, and microparticle density.

The levitation height of the cells or particles in a flowcell cartridge depends on the position of the flowcell cartridge relative to the magnets of the magnetic levitation system. As a result, the flowcell cartridge can be moved with respect to the magnets in order to capture cells that are levitating at various positions as a result of tagging them with density-modifying agents. As discussed in detail in the present disclosure, density-modifying non-magnetic microparticles may be used to segregate, by magnetic levitation, specified cells from a mixed population of cells suspended in a paramagnetic medium, resulting in fractions enriched or depleted for the cell type of interest. These fractions can be then withdrawn from the flowcell of the magnetic levitation system, accomplishing separation of a cell type of interest. As also described in the present disclosure, density-modifying non-magnetic microparticles may be used to separate, by magnetic levitation various components of interest from various types of heterogeneous mixtures, such as separation of cell organelles, nucleic acids, or other molecules. Components of interest (e.g., cells, cell organelles, nucleic acids) are sometimes referred to as “analytes.”

Thus, the present disclosure describes various embodiments of methods, devices, systems, and kits for magnetic levitation-based separation of mixtures or populations of particles that include various types of particles. Processes, devices, methods, and kits conceived by the inventors are useful for a variety of applications. Some embodiments of methods, devices, systems, and kits described in the present disclosure are useful for magnetic levitation-based separation of cells or subpopulations of cells from heterogeneous mixtures or populations of cells that include various cell types. Some other embodiments of methods, devices, systems, and kits described in the present disclosure are useful for magnetic levitation-based separation of mixtures or population of cellular organelles or other cellular components (including endocellular and exocellular components, for example, but not limited to, endosomes or exosomes). Yet some other embodiments of methods, devices, systems, and kits described in the present disclosure are useful for magnetic levitation-based separation of mixtures or population of biological molecules or complexes of molecules, such as separation of nucleic acids, for example, separation of nucleic acid libraries during next generation sequencing (NGS), or separation of lipoproteins.

Methods, devices, systems, and kits described in the present disclosure possess various advantages over previously known magnetic levitation-based separation methods.

Some of these advantages are improved separation precision and speed, improved reproducibility, and the ability to separate complex multi-component mixtures. Some other advantages are the ability to levitate molecules that would otherwise be difficult to levitate to a specific position during magnetic levitation, one example being RNA (such as RNA released from lysed cells), and the ability to separate, by magnetic levitation, cell types that have very similar densities (and therefore cannot be separated using magnetic levitation without the assistance of density-modifying non-magnetic microparticles described in the present disclosure).

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry are those well-known and commonly used. Known methods and techniques are generally performed according to conventional methods well-known and as described in various general and more specific references, unless otherwise indicated. The nomenclatures used in connection with the laboratory procedures and techniques described in the present disclosure are those well-known and commonly used.

Miscellaneous

As used in the present disclosure, the terms “a”, “an”, and “the” can refer to one or more unless specifically noted otherwise.

The use of the term “or” is used to mean “and/or,” unless explicitly indicated to refer to alternatives only, or the alternatives are mutually exclusive, although the disclosure supports a definition that refers to only alternatives and “and/or.” As used in the present disclosure “another” can mean at least a second or more.

As used in the present disclosure, and unless otherwise indicated, the terms “include,” “including,” and, in some instances, similar terms (such as “have” or “having”) mean “comprising.”

When a numerical range is provided in the present disclosure, the numerical range includes the range endpoints unless otherwise indicated. Unless otherwise indicated, numerical ranges in the present disclosure include all values and subranges, as if explicitly written out.

The terms “about” and “approximately,” as used in the present disclosure, shall generally mean an acceptable degree of error for the quantity measured, given the nature or precision of the measurements. Exemplary degrees of error are within 20%, 10%, 9%, 8%, 7%, 6%, 5%, 4%, 3%, 2%, or 1% of a given value or range of values. For example, any reference to “about X” or “approximately X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. In another example, the terms “about” or “approximately” in relation to a reference numerical value can include a range of values plus or minus 10%, 9%, 8%, 7%, 6%, 5%, 4%, 3%, 2%, or 1% from that value. Thus, expressions “about X” or “approximately X” are intended to describe a claim limitation of, for example, “0.98X.” Numerical quantities given in the present disclosure are approximate unless stated otherwise, meaning that the term “about” or “approximately” can be inferred when not expressly stated. When the terms “about” or “approximately” are applied to the beginning of a numerical range, they apply to both ends of the range. Where a series of values is prefaced with the terms “about” or “approximately,” these terms are intended to modify each value included in the series.

The terms “plurality” or “population,” when used in connection with particles, such as, but not limited to, cells (for example, as in “a plurality of cells” or “a population of cells”), refer to groups of particles (that is, more than one particle) including various numbers of particles. For example, a plurality or a population of particles, such as cells, may include 2 or more, 10 or more, 100 or more, 500 or more, 10³ or more, 10⁴ or more, 10⁵ or more, 10⁶ or more, or 10⁷, or more particles.

The terms “peptide,” “polypeptide” or “protein” are used to refer polymer of amino acids linked by native amide bonds and/or non-native amide bonds. Peptides, polypeptides or proteins may include moieties other than amino acids (for example, lipids or sugars). Peptides, polypeptides or proteins may be produced synthetically or by recombinant technology.

The terms “oligonucleotide,” “polynucleotide” or “nucleic acid” encompass DNA or RNA molecules, including the molecules produced synthetically or by recombinant technology. Oligonucleotides, polynucleotides or nucleic acids may be single-stranded or double-stranded.

The term “small molecule” includes molecules (either organic, organometallic, or inorganic), organic molecules, and inorganic molecules, respectively, which have a molecular weight of more than about 50 Da and less than about 2500 Da. Small organic (for example) molecules may be less than about 2000 Da, between about 100 Da to about 1000 Da, or between about 100 Da to about 600 Da, or between about 200 Da to about 500 Da.

Separation, Isolation, and Concentration

As used in the present disclosure, the term “concentration” means an amount of a first component contained within a second component, and may be based on the number of particles per unit volume, a molar amount per unit volume, weight per unit volume, or based on the volume of the first component per volume of the combined components.

As used in the present disclosure, the terms “isolate,” “separate, “segregate,” “purify,” and their respective related terms and expressions may be used interchangeably. These terms may be used to refer to a procedure that enriches the amount of one or more components of interest relative to one or more other components present in a sample. In reference to a particle or a component (which may be a cell), such terms may mean one or more of: separating such component from other components, increasing the concentration of a component within a solution, or separating a component from other components in a solution. For example, a particle within a solution may be deemed “isolated,” if it is segregated from other particles within the solution and/or positioned within a defined portion of the solution. In another example, a particle or component within a solution is deemed “isolated,” if, after processing the solution, the concentration of such particle or component is increased by a ratio of at least about 100:1, 90:1, 80:1, 70:1, 60:1, 50:1, 40:1, 30:1, 20:1, 10:1, 5:1, 3:1 2:1, 1.5:1 or 1.1:1. Particles of interest within a solution containing multiple types of particles may be deemed “separated” if, after processing the solution, the ratio of the concentration of the particles of interest to the concentration of other types of particles is increased, or if the ratio of the concentration of the particles of interest to the concentration of other types particles is increased by at least about 10%, 50%, 100%, 200%, 300%, 400%, 500%, 600%, 700%, 800%, 900%, or 1000%, or if the concentration of other components of the solution (including, but not limited to, the types of particles other than the particles of interest) is decreased to at least about 80%, 70%, 60%, 50%, 40%, 30% 20%, 15%, 10%, 8%, 6%, 5%, 4%, 3%, 2%, 1%, or 0.5%.

Density Modifying Agent

A density modifying agent comprises (i) a non-magnetic microparticle and (ii) a linking agent that preferentially binds to cells of the specified (e.g., first) type. The density modifying agent, by virtue of the linking agent, similarly preferentially binds to cells of the specified (e.g., first) type. Density modifying agents are sometimes referred to as “tags.” The process of linking density modifying agents to cells (or other objects) is sometimes referred to as “tagging.”

Non-Magnetic Microparticle

The terms “microparticle,” “microsphere,” “microbead,” and “microbubble,” (which encompass, respectively, a “nanosphere,” “nanosphere,” “nanoparticle,” “nanobead,” and “nanobubble”) refer to non-magnetic particles having one or more dimensions (such as length, width, diameter, or circumference) of about 500 μm or less (as discussed below). Microparticles may be described as having a characteristic density, as described below. A microparticle may have a generally spherical shape or a non-spherical shape. “Microbubbles” and “microbeads” are particular subtypes of microparticles.

The term “microbubble” usually refers to a microparticle that has an empty space or cavity (or multiple empty spaces or cavities) inside, the total volume of the empty spaces or cavities occupying an appreciably large (at least 50%, at least 60%, at least 70%, at least 80%, at least 90%, or at least 95%) proportion of the internal volume of the microparticle. The empty space or cavity (or multiple empty spaces or cavities) may be filled with gas. A microbubble may be described as “hollow.”

The term “microbead,” in the context of the present disclosure, usually refers to a microparticle that does not have appreciably large empty space or cavity (or multiple empty spaces or cavities) inside, the total volume of the empty spaces or cavities occupying no more than 50%, 40%, 30%, 20%, 10% or 5% of the internal volume of the microbead. A microbead may be described as “solid” or “substantially solid” (in the sense of not having appreciably large internal cavity or cavities). In the context of the present disclosure, a microbead may also be referred to as an “anchor.”

Microparticles used in the embodiments of the present invention can have range of sizes and densities. For example, a microparticle may have a cross-sectional dimension (e.g., diameter, length, width) about 500 μm or less, about 100 μm or less, about 50 μm or less, about 20 μm or less, about 10 μm or less, about 5 μm or less, about 1 μm (1000 nm) or less, about 0.5 μm (500 nm) or less, about 0.25 μm (250 nm) or less, about 0.1 μm (100 nm) or less about 0.05 μm (50 nm) or less, or about 0.025 μm (25 nm) or less, such as a cross-sectional dimension in the range of about 500 μm to about 0.01 μm (10 nm), about 500 μm to about 0.025 μm (25 nm), about 500 μm to about 0.05 μm (50 nm), about 500 μm to about 0.1 μm (100 nm), about 500 μm to about 0.25 μm (250 nm), about 500 μm to about 0.5 μm (500 nm), about 500 μm to about 1 μm (1000 nm), about 500 μm to about 10 μm, about 500 μm to about 20 μm, about 500 μm to about 100 μm, about 100 μm to about 0.01 μm (10 nm), about 100 μm to about 0.025 μm (25 nm), about 100 μm to about 0.05 μm (50 nm), about 100 μm to about 0.1 μm (100 nm), about 100 μm to about 0.25 μm (250 nm), about 100 μm to about 0.5 μm (100 nm), about 100 μm to about 1 μm (1000 nm), about 100 μm to about 10 μm, about 100 urn to about 20 μm, about 20 urn to about 0.01 urn (10 nm), about 20 urn to about 0.025 μm (25 nm), about 20 μm to about 0.05 μm (50 nm), about 20 μm to about 0.1 μm (100 nm), about 20 μm to about 0.25 μm (250 nm), about 20 μm to about 0.5 μm (20 nm), about 20 μm to about 1 μm (200 nm), or about 20 μm to about 10 μm.

Microparticles used in the embodiments of the present invention can be composed of, or can comprise, any number of materials or their combinations, including, but not limited to, glass, silica, ceramics, non-magnetic metals (such as gold, silver, or platinum), or lipids. In some embodiments, a microparticle is a polymeric microparticle. Polymeric microparticles can comprise or be composed of various types of polymers, such as, but not limited to, polymethyl methylacrylate (PMMA), polystyrene, polypropylene, polyethylene, polyacrylamide, alginic acid and its salts, or agarose. Some non-limiting examples of microparticles comprising combinations of materials are gold-coated silica particles and silica-coated gold particles. It is understood that microparticles can comprise various other combinations of two or more materials.

Microparticles may have a range of densities. More particularly, density modifying agent may have a range of densities, but it is contemplated that the linker portion will have a relatively small effect on the density of a density modifying agent, such that the microparticle density serves as a reasonable approximation of the density of a density modifying agent. It will be appreciated that all references to the density of a microparticle will refer equally to the density of a density modifying agent. Microparticles having densities in the following ranges may be used in the separation methods described herein: from about 0 g/cm³ to about 21.5 g/cm³, from about 0 g/cm³ to about 19.5 g/cm³, from about 0.001 g/cm³ to about 21.5 g/cm³, from about 0.001 g/cm³ to about 19.5 g/cm³, from about 0.01 g/cm³ to about 21.5 g/cm³, from about 0.01 g/cm³ to about 19.5 g/cm³, from about 0.1 g/cm³ to about 21.5 g/cm³, from about 0.1 g/cm³ to about 19.5 g/cm³, from about 0.1 g/cm³ to about 21.5 g/cm³, from about 0.1 g/cm³ to about 19.5 g/cm³, from about 0.5 g/cm³ to about 21.5 g/cm³, from about 0.5 g/cm³ to about 19.5 g/cm³, from about 0.7 g/cm³ to about 19.5 g/cm³, from about 0.7 g/cm³ to about 21.5 g/cm³, from about 0.9 g/cm³ to about 21.5 g/cm³, from about 0.9 g/cm³ to about 19.5 g/cm³, from about 0 g/cm³ to about 0.9 g/cm³, from about 0.001 g/cm³ to about 0.9 g/cm³, from about 0.01 g/cm³ to about 0.9 g/cm³, from about 0.1 g/cm³ to about 0.9 g/cm³, from about 0.5 g/cm³ to about 0.9 g/cm³, from about 0.7 g/cm³ to about 0.9 g/cm³, from about 1.2 g/cm³ to about 21.5 g/cm³, from about 2 g/cm³ to about 21.5 g/cm³, from about 3 g/cm³ to about 21.5 g/cm³, from about 4 g/cm³ to about 21.5 g/cm³, from about 5 g/cm³ to about 21.5 g/cm³, from about 6 g/cm³ to about 21.5 g/cm³, from about 7 g/cm³ to about 21.5 g/cm³, from about 8 g/cm³ to about 21.5 g/cm³, from about 9 g/cm³ to about 21.5 g/cm³, from about 10 g/cm³ to about 21.5 g/cm³, from about 11 g/cm³ to about 21.5 g/cm³, from about 12 g/cm³ to about 21.5 g/cm³, from about 13 g/cm³ to about 21.5 g/cm³, from about 14 g/cm³ to about 21.5 g/cm³, from about 15 g/cm³ to about 21.5 g/cm³, from about 16 g/cm³ to about 21.5 g/cm³, from about 17 g/cm³ to about 21.5 g/cm³, from about 18 g/cm³ to about 21.5 g/cm³, from about 19 g/cm³ to about 21.5 g/cm³, or from about 20 g/cm³ to about 21.5 g/cm³.

It will be recognized that the microparticles used in a single separation process may include multiple (two or more) microparticles with different densities. For example, one density microparticle may be coupled to a first cell type (e.g., CD8+ T cells) and a different density microparticle may be coupled to a second cell type (e.g., CD4+ T cells). In general, in a single cell separation process, density modifying agents that comprise the same linking agent (or comprise linking agents with the same specificity) will be associated with microparticles with the same density.

Factors Affecting Microparticle Selection

Binding of a density-modifying agent to cells or other objects and behaviors of the resulting complexes during magnetic levitation depends on several interconnected variables including, but not limited to, the density of the microparticles included in the density-modifying agent, the ratio of the microparticles to cells during complex formation (PTC ratio is discussed elsewhere in the present disclosure), microparticle size, the size of cells or other objects being tagged, the ratio of the above sizes, the density of the linking agent on the microparticle surface, and microparticle material. The above and other factors affect microparticle selection for a particular separation process and may be estimated using theoretical calculations, some of which are discussed below.

In one example, the density of a complex of a cell is estimated as the sum of the mass of the cell (m_{cell}) and all bound microparticles (n*m_{MS}) divided by the sum of the volume of the cell (V_{cell}) and all bound beads (n*V_{MS}).

$${ρ}_{complex} = \frac{{m}_{c e l l} + n * {m}_{M P}}{{V}_{c e l l} + n * {V}_{M P}}$$

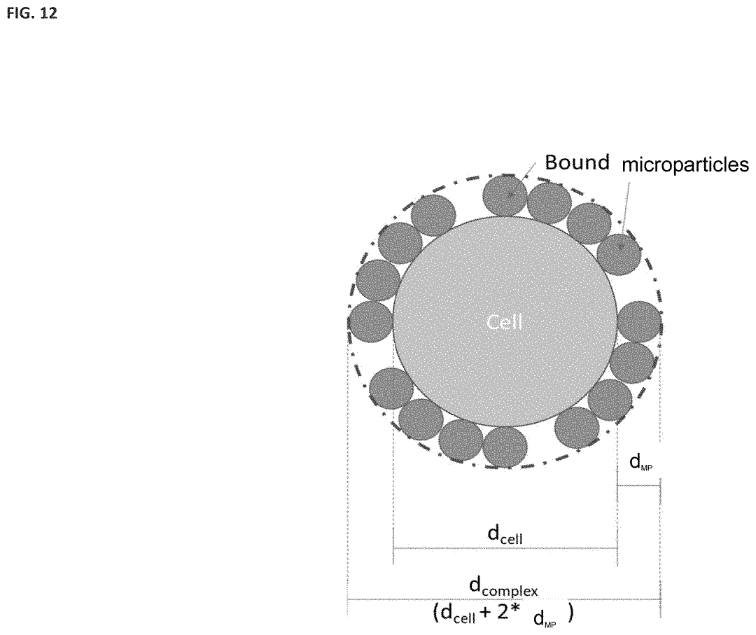

To estimate the theoretical maximum number of microparticles that can be bound to a cell, microparticles and cells are modeled as hard spheres forming a single layer of beads around the surface of the cell, as illustrated in FIG. 12. The volume available for the microparticles to occupy on the cell surface is determined by subtracting the volume of the cell from the volume of a sphere with diameter equal to twice the diameter of the microparticles (d_{MP}) plus the diameter of the cell (d_{cell}). The diameter of the complex is denoted d_{complex} in FIG. 12.

$${V}_{s h e l l} = {V}_{complex} - {V}_{c e l l}$$

$${V}_{c e l l} = 4 / 3 {π ( \frac{{d}_{c e l l}}{2} )}^{3}$$

$${V}_{complex} = \frac{4}{3} {π ( \frac{{d}_{c e l l} + 2 * {d}_{M P}}{2} )}^{3}$$

$$Max microparticles bound = 0.64 * ( \frac{{V}_{s h e l l}}{{V}_{M P}} )$$

In the above calculation, the volume of the shell around the cell is multiplied by a spherical packing factor of 0.64 (random packing of equal spheres) and divided by the volume of a single microsphere to determine the number of microsphere that are able to bind to the cell. The packing factor assumes random packing of spheres. This may be a conservative estimate of how many spheres may be packed around a larger central sphere.

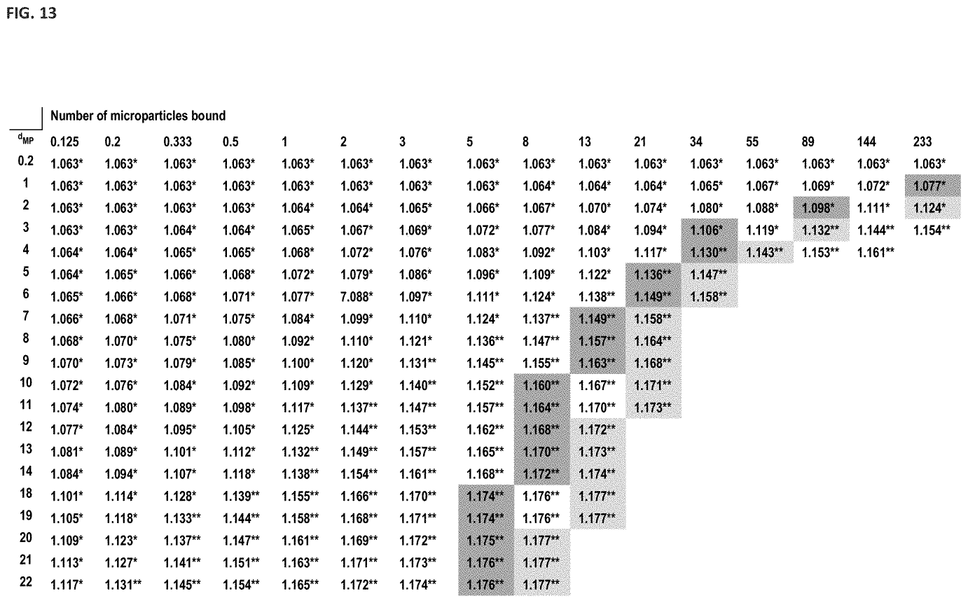

With a maximum number of microparticles that can fit around a cell established as an upper limit, estimates of microparticle-cell complex densities can be determined for various sizes of beads. For example, the table shown in FIG. 13 depicts the theoretical densities (g/cm³) of microparticle-cell complexes with various sizes of microparticles over a range of microparticle diameters and the numbers of microparticles bound per cell. The microparticles used in the calculation were assigned the density of 1.18 g/cm³. The cells used in the calculation were assigned the density of 1.063 g/cm³ and a diameter of 11.5 μm. In the table shown in FIG. 13, the table cells highlighted in darker grey indicate a complex with about 40-60% microparticle coverage of the cell surface. The table cells highlighted in lighter grey indicate a complex with about 80-100% microparticle coverage of the cell surface. The complexes with densities less than or greater than 1.13 g/cm³ are labeled with single or double asterisks, respectively. The value of 1.13 g/cm³ was chosen for the specific calculation illustrated in FIG. 13, because it represented a cut-off density for the specific conditions of an exemplary magnetic levitation experiment. The complexes with the density below the cut-off would be collected in the bottom portion of the processing channel of the flowcell cartridge, and thus separated from the complexes with the densities above the cut-off, which would be levitating higher in the top portion of the processing channel of the flowcell cartridge. Other cut-off values may be used, depending on the particular experimental conditions and desired outcomes. The calculation illustrated in FIG. 13 estimates that, for larger microparticles, fewer microparticles per cell are needed to increase the density of the complex above the chosen cut-off, but there is also less available space to fit those larger microparticles on the cell surface. As determined by the calculation illustrated in FIG. 13, the microparticles with diameter of less 3 μm would not form a complex with the density above the chosen cut-off value, because it would require >100% coverage of the cell surface by the microparticles. Based on the illustrated calculation, the microparticles with the diameter of about 4-5 μm can achieve the density above the chosen cut-off value with about 50% coverage of the cell surface.

The calculation illustrated in FIG. 13 estimates the number of microparticles per cell in a complex to achieve a specific density. Given that the binding kinetics of the density modifying agent are largely driven by the ligand-receptor binding interactions, a higher ratio of the microparticles to cells during complex formation (PTC ratio discussed elsewhere in the present disclosure) needed to achieve a target ratio of microparticles per cell in the complex. To satisfy equilibrium binding mechanics, steric hindrance, and diffusion of microparticles through solution to interact with the cells, the effective concentration of a linking agent included in the density modifying agent (which comprises a microparticle and a linking agent) needs to be maximized during complex formation, for example, by selecting the microparticles of the smaller diameter, in order to be able to increase the number of microparticles during complex formation. Another factor that needs to be taken into consideration is the product of the number of linking agent molecules bound to the surface of each microparticle and the total microparticles in suspension. For example, a microparticle with a mean diameter of 150 nm, 50% streptavidin coverage of each particle, and four binding sites per streptavidin molecule, would provide 1×10⁻¹⁴ μmol concentration of biotinylated antibody bound to streptavidin. For a sample of 2×10⁵ cells in 100 μl, a microparticle-to-cell ratio of 50,000:1 would yield about 1 μm concentration of the antibody, which is 10-100× the dissociation constant (K_{D}) of typical antibody interactions (10-100 nm). In comparison, a microparticle with a mean diameter of 5 μm at a microparticle-to-cell ratio of 40:1, with all the other conditions held the same, would still yield about 900 nm concentration of antibody. However, in this scenario, the amount of antibody attached to each microparticle is about 1000× as high. This limits access of the antibody to the cells during complex formation, as all the available antibodies are localized to a smaller total number of microparticles. It is envisioned that, for higher affinity linking agents, lower PTC ratios are required for effective complex formation, since the interaction between the linking agent and the cell is more likely to persist. Lower affinity linking agents likely require higher PTC ratios for effective complex formation to achieve and maintain the target microparticle-per-cell number in the complexes.

Manufacture and Sourcing of Microparticles

Some methods of producing microparticles are described, for example, in Lu et al. “Modular and Integrated Systems for Nanoparticle and Microparticle Synthesis—A Review” Biosensors (2020) 10(11):165. https://doi.org/10.3390/bios10110165. Commercially available microparticles can also be used. Some exemplary microparticle sources are Nanopartz (Loveland, Colo., USA), Cospheric (Santa Barbara, Calif., USA), Creative Diagnostics (New York, N.Y., USA), Spherotech (Lake Forest, Ill., USA), Bangs Laboratories (Fishers, Ind., USA), PolyAn (Berlin, Germany), Polysciences (Warrington, Pa., USA), and Lab261 (Palo Alto, Calif., USA).

Linking Agent

A “linking agent” is used to couple a non-magnetic microparticle(s) to a component of interest (e.g., a cell of interest, organelle, nucleic acids). A linking agent specifically binds to the cell or other analyte. One example of a linking agent is an antibody that specifically binds to a cell surface protein displayed on a cell of interest. Other types of linking agents include aptamers, ligands (that are bound by a cell-surface receptor), including, but not limited to, small molecule ligands and polypeptide or protein ligands, lipophilic tags, and nucleic acids (at least a portion of which is complementary to a target nucleic acid). For example, removal of mRNA from a sample can be performed by coupling an Oligo-dT to microparticles, which then bind to the poly-A tail at the end of mRNA. In another example, total RNA can be removed from a sample by coupling random hexamer oligonucleotides to microparticles, which then bind to random RNA hexanucleotides.

Antibody

The term “antibody” and the related terms, in the broadest sense, are used in the present disclosure to denote any product, composition or molecule that contains at least one epitope binding site, meaning a molecule capable of specifically binding an “epitope”—a region or structure within an antigen. The term “antibody” encompasses whole immunoglobulin (i.e., an intact antibody) of any class, including natural, nature-based, modified, and non-natural (engineered) antibodies, as well as their fragments. The term “antibody” encompasses “polyclonal antibodies,” which react against the same antigen, but may bind to different epitopes within the antigen, as well as “monoclonal antibodies” (“mAbs”), meaning a substantially homogenous population of antibodies or an antibody obtained from a substantially homogeneous population of antibodies. The antigen binding sites of the individual antibodies comprising the population of mAbs are comprised of polypeptide regions similar (although not necessarily identical) in sequence. The term “antibody” also encompasses fragments, variants, modified and engineered antibodies, such as those artificially produced (“engineered), for example, by recombinant techniques. For instance, the term “antibody” encompasses, but is not limited to, chimeric antibodies and hybrid antibodies, antibodies with dual or multiple antigen or epitope specificities, and fragments, such as F(ab′)2, Fab′, Fab, hybrid fragment, single chain variable fragments (scFv), “third generation” (3G) fragments, fusion proteins, single domain and “miniaturized” antibody molecules, and “nanobodies.”

Aptamer

Nucleic acid aptamers are RNA or single stranded DNA molecules, which can fold into various architectures and bind to a wide array of targets including other nucleotides or proteins. For example, aptamers to tumor cell-surface markers, including HER-2, a breast cancer cell surface marker, may be used for isolating or removing tumor cells from a tissue sample. Aptamers Targeting Tumor Cell-Surface Protein Biomarkers, as well as selection of such aptamers, are discussed, for example, in Mercier et al., Cancers (Basel) 9(6):69 (2017) doi: 10.3390/cancers9060069.

Lipophilic Tag

The linking agent may be a lipophilic tag. Lipophilic tags are lipophilic molecules that can associate with and/or insert into lipid membranes such as cell membranes and organelle membranes. Examples of lipophilic molecules include sterol lipids (e.g., cholesterol or tocopherol), steryl lipids, lignoceric acid, and palmitic acid. By themselves, lipophilic tags do not accomplish specific binding. However, they may be used to specifically target cell membranes in the mixtures of cells with other particles. In addition, different samples can be tagged with differentially-labeled lipophilic tags.

Other Specific Binding Partners

The expression “specific binding molecule” denotes a molecule capable of specifically or selectively binding another molecule or a region or structure within another molecule, which may be termed “target,” “ligand” or “binding partner.” The terms “specific binding,” “selective binding” or related terms refer to a binding reaction in which, under designated conditions, a specific binding molecule or a composition containing it binds to its binding partner or partners and does not bind in a significant amount to anything else. Binding to anything else other than the binding partner is typically referred to as “nonspecific binding” or “background.” The absence of binding in a significant amount is considered, for example, to be binding less than 1.5 times background (i.e., the level of non-specific binding or slightly above non-specific binding levels). Some non-limiting examples of specific binding are antibody-antigen or antibody-epitope binding, binding of oligo- or polynucleotides to other oligo- or polynucleotides, binding of oligo- or polynucleotides to proteins or polypeptides (and vice versa), binding or proteins to polypeptides other proteins or polypeptides, receptor-ligand binding, and carbohydrate-lectin binding. Accordingly, specific binding molecules can be or can include a protein, a polypeptide, an antibody, an oligo- or polynucleotide, a receptor, or a ligand. Specific binding molecules can be natural or engineered. For example, both engineered and naturally occurring nucleic acid or peptide aptamers can serve as specific binding molecules in the embodiments of the present invention. This list is not intended to be limiting, and other types of specific binding molecules may be employed. The term “target molecule” is used to denote a molecule or a part thereof, including a biological molecule (such as, but not limited to, a protein, a peptide, lipid, a nucleic acid, a fatty acid, or a carbohydrate molecule, such as an oligosaccharide), or a nonbiological molecule (including a small molecule, such a small molecule drug or a small molecule ligand). A specific binding molecule, such as antibody, specifically binds to the target molecule.

Indirect Binding

Specific binding molecules, such as, but not limited to, antibodies, can be directly attached to density-modifying microparticles, for example, by surface conjugation, coating, or adsorption. However, specific binding molecules need not be directly attached to density-modifying microparticles, and can be used for complexing density-modifying molecules with a target cell via an intermediary non-covalent binding interaction. For example, specific binding molecule is a biotinylated antibody capable of specifically binding to a target cell, and density-modifying microparticles are coated with avidin, streptavidin, neutravidin, or any form of modified avidin, which can be referred to as “avidin-like compound.” An intermediary binding interaction between an avidin-like compound on the density-modifying microparticles and biotin moiety of the antibody allows for formation of a complex between a target cell and a density-modifying microparticle. In another example, specific binding molecule is an antibody capable of specifically binding to a target cell (“primary antibody”), and density-modifying microparticles are coated with protein A, protein S, or an anti-antibody (that is, an antibody against primary antibody). An intermediary binding interaction between protein A, protein S, or an anti-antibody on the density-modifying microparticles and the primary antibody allows for formation of a complex between a target cell and a density-modifying microparticle.

Fluidics

As used in the present disclosure, the term “fluidic” refers to a system, device or element for handling, processing, ejecting and/or analyzing a fluid sample including at least one “channel” as defined elsewhere in the present disclosure. The term “fluidic” includes, but is not limited to, microfluidic and nanofluidic.

As used in the present disclosure, the terms “channel”, “flow channel,” “fluid channel” and “fluidic channel” are used interchangeably and refer to a pathway on a fluidic device in which a fluid can flow. Channel includes pathways with a maximum height dimension of about 100 mM, about 50 mM, about 30 mM, about 25 mM, about 20 mM, about 15 mM, about 10 mM, about 5 mM, about 3 mM, about 2 mM, about 1 mM, or about 0.5 mM. For example, the channel between magnets can have cross-sectional dimensions (height by width) of about 10 mM×10 mM, about 10 mM×5 mM, about 10 mM×3 mM, about 10 mM×2 mM, about 10 mM×1 mM, or about, about 10 mM×0.5 mM, about 5 mM×10 mM, about 5 mM×5 mM, about 5 mM×3 mM, about 5 mM×2 mM, about 5 mM×1 mM, about 5 mM×0.5 mM, about 3 mM×10 mM, about 3 mM×5 mM, about 3 mM×3 mM, about 3 mM×2 mM, about 3 mM×1 mM, about 3 mM×0.5 mM, about 2 mM×10 mM, about 2 mM×5 mM, about 2 mM×3 mM, about 2 mM×2 mM, about 2 mM×1 mM, about 2 mM×0.5 mM, about 1 mM×10 mM, about 1 mM×5 mM, about 1 mM×3 mM, about 1 mM×2 mM, about 1 mM×1 mM, about 1 mM×0.5 mM, about 0.5 mM×10 mM, about 0.5 mM×5 mM, about 0.5 mM×3 mM, about 0.5×2 mM, about 0.5 mM×1 mM, or about 0.5 mM×0.5 mM. The internal height of the channel may not be uniform across its cross-section, and geometrically the cross-section may be any shape, including round, square, oval, rectangular, or hexagonal. The cross-section may vary along the length of the channel. The term “channel” includes, but is not limited to, microchannels and nanochannels, and, with respect to any reference to a channel in the present disclosure, such channel may comprise a microchannel or a nanochannel.

As used in the present disclosure, the term “fluidically coupled” or “fluidic communication” means that a fluid can flow between two components that are so coupled or in communication.

Magnetic Levitation

The expression “magnetic levitation,” in the context of the present disclosure and as described, for example, in U.S. patent application Ser. No. 14/407,736, generally involves subjecting diamagnetic, paramagnetic, ferromagnetic, ferrimagnetic or antiferromagnetic materials or “objects” suspended in a paramagnetic fluid medium to a magnetic field, such as a magnetic field gradient that forms between two magnets. The magnetic field generates a non-uniform pressure equivalent to the magnetic energy density in the paramagnetic fluid medium. In a magnetic field gradient, the objects appear to be repelled from the regions of high magnetic field. In actuality, the object is displaced by an equal volume of the paramagnetic fluid medium. The attractive interaction between the paramagnetic fluid medium and the regions of high magnetic field can result in the “levitation” of the object. The “levitation height” of an object, in the two-magnet setup, can be defined as desired. For example, in certain embodiments, “levitation height” can be defined as the distance between the center of the levitating object and the top surface of the bottom magnet, but any desired reference point can be utilized. By applying the magnetic field in such a manner that the force on the objects is opposed by another uniform force (e.g., the force of gravity), a balance is achieved for the object that is directly related to its density.

Paramagnetic Fluid Medium

In the context of the present disclosure, a “paramagnetic fluid medium” may a paramagnetic material and a solvent. In some embodiments of the methods described in the present disclosure, the paramagnetic fluid medium is biocompatible, i.e. capable of being mixed with live cells and not impact the viability of the cells or impacting cellular behavior. A paramagnetic material may include one or more of: gadolinium, titanium, vanadium, dysprosium, chromium, manganese, iron, nickel, gallium, including their ions. For example, a paramagnetic material may include one or more of the following ions: titanium (III) ion, gadolinium (III) ion, vanadium (I) ion, nickel (II) ion, chromium (III) ion, vanadium (III) ion, dysprosium (III) ion, cobalt (II) ion, and gallium (III) ion. In some embodiments, a paramagnetic material comprises a chelated compound, such as, but not limited to, a gadolinium chelate, a dysprosium chelate, or a manganese chelate. In some examples, a paramagnetic material may comprise one or more of [Aliq]₂ [MnCl₄], [Aliq]₃ [GdCl₆], [Aliq]₃ [HoCl₆], [Aliq]₃ [HoBr₆], [BMIM]₃ [HoCl₆], [BMIM] [FeCl₄], [BMIM]₂ [MnCl₄], [BMIM]₃ [DyCl₆], BDMIM]3 [DyCl₆], [AlaC1] [FeCl₄], [AlaCl]₂ [MnCl₄], [AlaCl]₃ [GdCl₆], [AlaCl]₃ [HoCl₆], [AlaCl]₃ [DyCl₆], [GlyC2] [FeCl₄]. In one exemplary embodiment, a paramagnetic material is gadobutrol. A paramagnetic material may be present in the paramagnetic fluid medium at a concentration of at least about 10 mM, 20 mM, 30 mM, 40 mM, 50 nm, 60 mM, 70 mM, 80 mM, 90 mM, 100 mM, 120 mM, 150 mM, 200 mM, 250 mM, 300 mM, 500 mM, 1 M, about 10 mM to about 50 mM, about 25 mM to about 75 mM, about 50 mM to about 100 mM, about 100 mM to about 150 mM, about 150 mM to about 200 mM, about 200 mM to about 250 mM, about 250 mM to about 300 mM, about 300 mM to about 500 mM, or about 500 mM to about 1 M. In an exemplary embodiment, a paramagnetic material comprises gadolinium, and the paramagnetic material is present in the paramagnetic fluid medium at a concentration of at least about 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, about 10 mM to about 50 mM, about 25 mM to about 75 mM, about 50 mM to about 100 mM, about 50 mM to about 200 mM, about 50 mM to about 300 mM, about 50 mM to about 400 mM, about 50 mM to about 500 mM, about 50 mM to about 600 mM, about 50 mM to about 700 mM, about 50 mM to about 800 mM, about 50 mM to about 900 mM, or about 50 mM to about 1 M. It is understood that, in addition to a paramagnetic material and a solvent, a paramagnetic fluid medium may comprise other components, such as salts or additives, for example, but not limited to, additives that function to maintain cellular integrity.

Magnetic Levitation Systems and Components

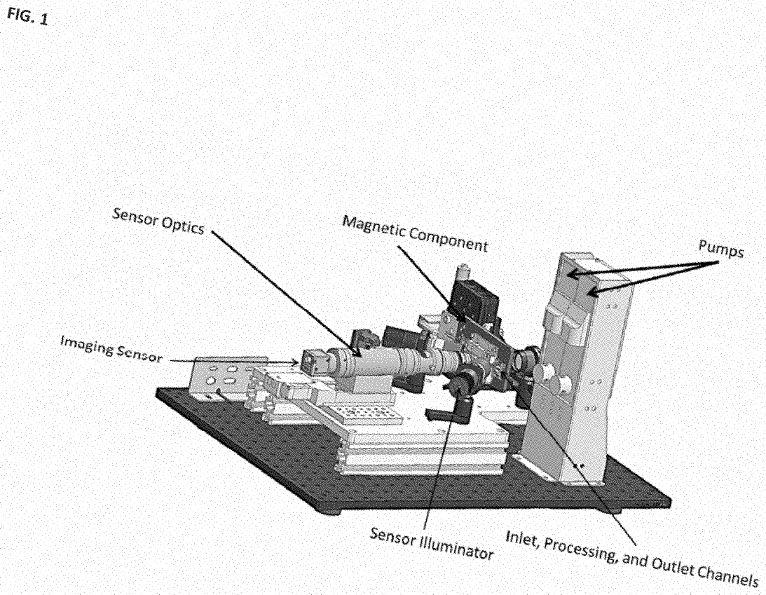

Exemplary magnetic levitation systems that and their components are described, for example, in Durmus et al. and in U.S. patent application Ser. No. 17/449,438, filed Sep. 29, 2021. While various embodiments of the invention provided in the present disclosure are not limited by any particular magnetic levitation system, a brief description is included to facilitate the understanding of the methods, kits and systems according to the embodiments of the present invention. An exemplary magnetic levitation system is schematically illustrated in FIG. 1.

Flowcell Cartridge

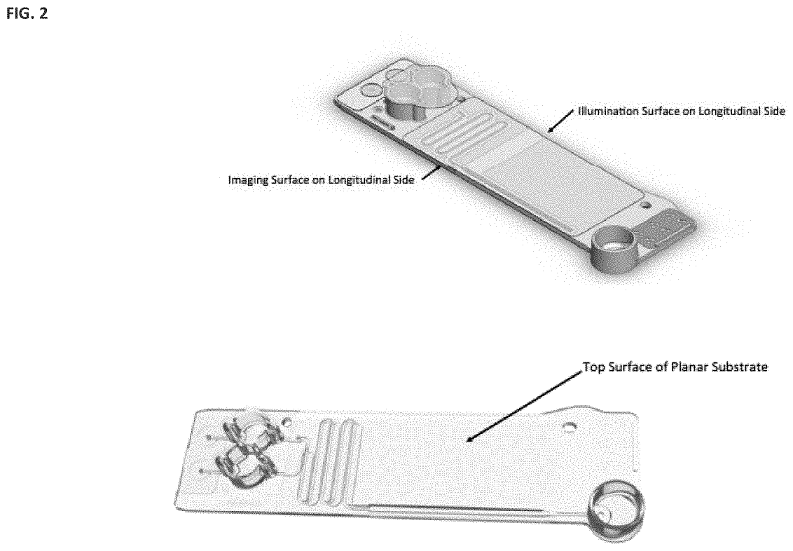

At least some methods and kits according to the embodiments of the present invention involve a flowcell cartridge for use in a magnetic levitation system. An exemplary flowcell cartridge is schematically illustrated in FIG. 2. An exemplary flowcell cartridge may include a planar substrate comprising an upper surface and a lower surface, a first longitudinal side forming an imaging surface, a second longitudinal side forming an illumination surface, and a first and second transverse side, an inlet well on an upper surface, an inlet channel, a sample processing channel in fluidic communication with the inlet channel and positioned substantially parallel to a longitudinal side, a sample splitter within the processing channel, a plurality of outlet channels in fluidic communication with the processing channel, and a plurality of collection wells in fluidic communication with each of the plurality of outlet channels. The planar configuration allows for all required flowcell cartridge functions to be integrated into the flowcell cartridge and increases performance and reproducibility in a laboratory or clinical setting. In operation, it is important for enhanced performance that the flow the processing channel and into the outlet channel be as free of turbulence as possible. The processing channel may be offset within the plane of the of the planar substrate to be spatially biased to the imaging surface.

The flowcell cartridge can be formed by injection molding, etching, laser ablation, machining, or 3D printing. When imaging within the flowcell cartridge is desired, the planar substrate comprises an optically transparent material. Glass, plastic, or polymer materials including cyclic olefin polymer (COP) or cyclic olefin copolymer (COC) are some examples of suitable optically transparent materials. Dimensions of the planar substrate can be at least 50 mM in length, 20 mM in width, and at least 1.5 mM in thickness. Optional ranges are at least 100 mM in length, 35 mM in width, and about 2 to about 6 mM in thickness. The longitudinal sides of the cartridge can act as waveguides for illumination and imaging. For that reason, the processing channel is offset in the plane of the substrate and is parallel and adjacent to the imaging longitudinal side of the substrate. Distances from the imaging side wall can be from about 0.5 mM to about 10 mM, preferably from about 0.5 mM to about 5 mM, optionally from about 1 mM to about 3.5 mM. In an embodiment the processing channel spacing from the imaging wall is about 2 mM. The volume of the processing channel can be from about 10 μL to about 800 μL, from about 50 μL to about 600 μL, 100 μL to about 400 μL, about 150 μL to about 300 μL, at least about 150 μL, at least about 200 μL, at least about 250 μL, or at least about 300 μL. The combined volume of the outlet channels can be greater that the volume of the processing channel. The flow volume split between two outlet channels can be an even split or can range from about 4:1 to about 1:4, about 3:1 to about 1:3 or about 2:1 to about 1:2 or can vary from 1:1 by about 50% or less, or about 40% or less, or about 30% or less, or about 15% or less when in operation in the system embodiments.

A flowcell cartridge optionally includes collection wells on the planar substrate. The collection wells feature an inlet that is in fluidic communication with the outlet channel. The inlet can be at a first well height and configured with a step transitioning from the inlet port aperture to the floor of the well. This provides a transition surface for the flow of sample fraction into the well and can inhibit back siphoning of the sample fraction into the outlet channel as well as bubble formation within the collection well. An outlet channel within the collection well can be provided with an opening that is at a height off the floor of the collection well that is higher than the opening of the inlet channel. The internal outlet can be placed in communication with a flow modulator. In some instances, the flow modulator is an individual pump to provide flow through the flowcell cartridge. In operation, the collection well is sealed with a layer of material or film to provide an enclosed system to allow flow or pumping of sample and sample fractions through the flowcell cartridge. In assembling the flowcell cartridge layers, and when an adhesive is used, a biocompatible adhesive can be used for biological applications. Correct adhesive selection is necessary to minimize or prevent leaching of adhesive components into the solution, adhering to cells or binding molecules from solution, being autofluorescent, having texture which increases the surface area and hence the impact on cells, and overly hydrophilic or hydrophobic. An example of a suitable adhesive is a silicone or silicone-based adhesive.

Separation System

An exemplary magnetic levitation-based separation system (illustrated in FIG. 1) comprises a receiving block for retaining a flowcell cartridge, an optical system comprising an optical sensor, a lens, and an illumination source, and plurality of flow modulation components. The receiving block removably places the flowcell cartridge in optical alignment with the optical system, removably engages a magnetic component adjacent to the processing channel of the flowcell cartridge, and removably places a plurality of outlet channels of the flowcell cartridge in fluidic communication with the plurality of flow modulation components. The optical system may be constructed to provide microscopic imaging of the processing channel of the flowcell cartridge. Optionally, the optical system may be constructed and arranged to provide imaging for florescence emission with optional ultraviolet light exciter modules. The optical system may comprise a source of visible optical illumination constructed and arranged to provide light transmission through the processing channel within the planar substrate. The receiving block is constructed and arranged to hold the planar flowcell cartridge in an orientation to the optical system, such that the imaging optics are aligned with the imaging side of the planar cartridge and the visible light emitter is in an orientation to illuminate the illumination side of the planar flowcell cartridge. Optionally, the optical system can further comprise one or more sources of ultraviolet or visible illumination constructed and arranged to place the ultraviolet illumination, in an angular orientation the imaging side of the planar cartridge to excite fluorophores within the processing channel for the cartridge. For imaging of fluorescent entities internal to the processing channel optical system optionally comprises a dual bandpass filter preferably passing emitted radiation in bands centered at wavelengths at about 524 nm and 628 nm.

An optional feature of the receiving block is a series of flow modulator adapters that interface with outlets on the top or bottom of the flowcell cartridge. The adapters facilitate fluidic communication with flow modulators, such as a pump in the system, with outlet channels of the flow cells such as the collection well outlet channels. Once the flowcell cartridge is inserted into the receiving block, the receiving block is mechanically actuated to support the cartridge, aligning the illumination and imaging sides of the planar cartridge with the optical imaging system, aligning the magnetic components to position them above and below the flowcell processing channel, and, where desired, place the flow modulator adapters in fluidic communication with corresponding outlet channels of the flowcell cartridge. The flow modulators of the system provide flow to the sample and sample fractions within the flowcell cartridge. The flow rate provided by the flow modulators can range from as low as 1 μL per minute to as high as 1 mL per minute during separations. The flow rate can be at or at least about 25 μL per minute, at or at least about 50 μL per minute, at or at least about 100 μl per minute, at or at least about 200 μL per minute, at or at least about 250 μL per minute, at or at least about 300 μL per minute, or from about 300 μL per minute to about 1 mL per minute. The total sample volume flowrate can be about 50 μL/min, about 75 μL/min, about 100 μL/min, about 150 μL/min, about 200 μL/min or about 300 μL/min. The flow volume split between two outlet channels can be an even split or can range from about 4:1 to about 1:4, about 3:1 to about 1:3 or about 2:1 to about 1:2 or can vary from 1:1 by about 50% or less, or about 40% or less, or about 30% or less, or about 15% or less when in operation in the system embodiments.

A magnetic levitation system is capable of magnetically levitating particles suspended in a paramagnetic fluid medium within a processing channel or inlet channel of a flowcell cartridge. The interaction of the magnetic field with the paramagnetic properties of particles within a paramagnetic fluid medium can either provide a repulsive or attractive effect on the particles to facilitate their separation or concentration. The magnetic field in a magnetic fluid medium is created by magnets, which can be permanent magnets or electromagnets. The maximum energy product of magnets can range from about 1 Mega-Gauss Oersted to about 1000 Mega-Gauss Oersted, or from about 10 Mega-Gauss Oersted to about 100 Mega-Gauss Oersted. The surface field strength of magnets can range from about 0.01 Tesla to about 100 Tesla, or from about 1 Tesla to about 10 Tesla. The remanence of magnets can range from about 0.5 Tesla to about 5 Tesla, or from about 1 Tesla to about 3 Tesla. Magnets can be made from a material comprising neodymium alloys with iron and boron, neodymium, alloys of aluminum with nickel, neodymium alloys with iron, aluminum and cobalt alloyed with iron, samarium-cobalt, other alloys of rare earth elements with iron, alloys of rare earth alloys with nickel, ferrite, or combinations thereof. When a magnetic levitation system comprises a plurality of magnets, magnets can be made from the same material or are made from different materials.

An asymmetric magnetic field can be achieved by using a stronger magnetic material on one side of a fluidic channel of a flowcell cartridge and a weaker magnetic material on the opposite side of the fluidic channel of a flowcell cartridge. An asymmetric magnetic field can be achieved by positioning a magnet closer on one side of a channel than a magnet on the other side. An asymmetric magnetic field can be achieved by using a magnetic material on one side of a fluidic channel of a flowcell cartridge and a substantially similar magnetic material on the opposite side of the fluidic channel of a flowcell cartridge. An upper magnet and a lower magnet may be substantially the same size. In one example, the upper magnet may comprise neodymium, the lower magnet may comprise samarium-cobalt. Alternatively, the upper magnet may comprise samarium-cobalt, and the lower magnet may comprise neodymium. Alternative magnet configurations may be used. A magnetic levitation system may include multiple upper magnets and multiple lower magnets positioned around a fluidic channel of a flowcell cartridge. In one example, upper magnets may include an anterior upper magnet, a central upper magnet, and a posterior upper magnet. Lower magnets may include an anterior lower magnet, a central lower magnet, and a posterior lower magnet. In another example, a magnetic levitation system may include an anterior upper magnet, a posterior upper magnet, an anterior lower magnet, and a posterior lower magnet, with the magnets positioned around a fluidic channel of a flowcell cartridge, and the anterior upper magnet and the posterior lower magnet are positioned in a magnetic repelling orientation. Exemplary NdFeB magnetic component dimensions include, for a bottom magnet component about 50×15×2 mM (magnetized through the 15 mM axis), 50×5×2 mM (magnetized through the 5 mM axis) for a top magnet component. Other exemplary magnet dimensions include 60×15×2 mM, 60×5×2 mM, 75×10×3 mM, 75×20×3 mM, and 25×15×2 mM. An exemplary magnet configuration of a magnetic levitation system includes an upper and lower magnet with dimensions of about 75×20×3.2 mM, and a spacing between upper and lower magnets of about 2.5 mM, about 3.0 mM, about 3.5 mM, about 2.9 mM, about, 3.0 mM, about 3.1 mM, about 3.2 mM, about 3.3 mM, or about 2.72 mM, about 2.88 mM, about 2.98 mM, about 3.18 mM, about 3.20 mM, or about 3.37 mM. One more exemplary magnet configuration of a magnetic levitation system has an upper magnet and a lower magnet, with the lower magnet extending into an inlet channel of a flowcell cartridge. The bottom magnet dimensions can be about 50 mM to about 100 mM×about 10 mM to about 30 mM×about 2 mM to about 4 mM.

Samples

The terms “sample” or “samples,” and the related terms and expressions, as used in the present disclosure, are not intended to be limiting, unless qualified otherwise. These terms refer to any product, composition, cell, tissue or organism. Generally, the terms “sample” or “samples” are not intended to be limited by their source, origin, manner of procurement, treatment, processing, storage or analysis, or any modification. Some examples of the samples are solutions, suspensions, supernatants, precipitates, or pellets. Samples can contain or be predominantly composed of cells or tissues, or can be prepared from cells or tissues. However, samples need not contain cells. Samples may be mixtures of or contain biological molecules, such as nucleic acids, polypeptides, proteins (including antibodies), lipids, carbohydrates etc. Samples may be biological samples. For example, a “sample” may be any cell or tissue sample or extract originating from cells, tissues or subjects, and include samples of animal cells or tissues as well as cells of non-animal origin, including plant and bacterial samples. A sample can be directly obtained from an organism, or propagated, or cultured. Some exemplary samples are cell extracts (for examples, cell lysates), suspensions of cell nuclei, liquid cell cultures, cell suspensions, biological fluids (including, but not limited to, blood, serum, plasma, saliva, urine, cerebrospinal fluid, amniotic fluid, tears, lavage fluid from lungs, or interstitial fluid), tissue sections, including needle biopsies, microscopy slides, frozen tissue sections, or fixed cell and tissue samples.

Exemplary Cell Types

Cells tagged with density modifying agents may be, for illustration and not limitation, human cells, non-human animal cells, plant cells, eukaryotic cells, etc. For example, tagged cells may be, but are not limited to, human or non-human immune cells, endothelial cells, and T-cells. Tagged cells may be in a heterologous population of untagged cells, which may be, for illustration and not limitation, human cells, non-human animal cells, plant cells, eukaryotic cells, prokaryotic cells, etc. Tagged cells may be from different lineages, or could be the same lineage but different in activation state, differentiation state, or some other property.

Tagging Cells

One or more than one (2, 3, 4, or more than 4) cell types may be processed in a single separation step. When two or more cell types are tagged, each cell type may be tagged with a different density modifying agent. Alternatively two or more different cell types may be tagged with the same density modifying agent. For example, two or more different cell types may have the same surface markers, and density modifying agent comprising the same linking agent may therefore bind to both cell types. In another example, a density modifying agent comprises two or more different linking agents that can bind to respective two or more surface markers. In this situation, the density modifying agent can bind to two or more different cell types with different surface markers corresponding to two or more different linking agents. In some embodiments, two or more different cell types may be tagged with different density modifying agents that have the same density.

Tagging Process

As a part of a tagging process, microparticles comprising a linking agent specific to a target cell type may mixed with the cells at a ratio optimized for the target cell type. A ratio of the microparticles to cells in the mixture (PTC ratio, discussed elsewhere in the present disclosure) is optimized based on various parameters. One of these parameters may be the target cell type, which determines its levitation profile, but may also affect the number of microparticles comprising a linking agent with which each cell may complex. For example, if the linking agent is specific for cell surface markers, the number of markers on a particular target cell type will determine how many units of the microparticles comprising the linking agent can bind to this cell. Another parameter may be the affinity of the linking agent, such as an antibody, for a particular cell type. One more parameter is the magnetic field strength applied during magnetic levitation. Other parameters that may be taken into account when determining PTC ratio may be microparticle size, and/or microparticle density. It is understood that other parameters, not listed here, may also be taken into account, and also that PTC ratio may be experimentally determined and/or optimized for a particular tagging and/or separation application. Non-limiting PTC ratios used in the embodiments of the methods described in the present disclosure may be (but are not limited to) from about 1 to about 100,000, from about 1 to about 50,000, from about 1 to about 10,000, from about 1 to about 1,000, from about 1 to about 100, from about 10,000 to about 100,000, from about 10,000 to about 50,000, or from about 50,000 to about 100,000, When multiple cell types are present, microparticles with different linking agents specific for each cell type may be mixed together prior to addition to cell mixture, or microparticles with different linking agents and cells will be mixed together in one step. In some cases, the tagging process may be performed serially on a cell mixture, mean that microparticles with different linking agents specific for each cell type may be applied after each tagging and separation step.

Design and Selection of Density Modifying Agent

Design and selection of density modifying agent or agents, as well as their amounts, used in the embodiments of the present invention take into account various scenarios. For example, some cells of the same type may be tagged with different numbers of the same density modifying agent. As a result, cells of the same type tagged with the same density-modifying agents may have different densities and levitation heights. In certain situations, this problem may be at least partially addressed by adjusting the ratio of a density-modifying agent and projected number of target cells in samples, as the difference in the numbers of a density-modifying agent tagging the target cell type may be more pronounced at some ratios more than the others. In another example, target cells with the same number of surface markers per surface area may be tagged with different numbers of density-modifying agent units. In certain situations, this problem may be at least partially addressed, for example, by increasing the ratio of density modifying agents and projected number of target cells in sample in an attempt to saturate all available surface markers. In other examples, this problem may be at least partially addressed by increasing the time of incubation of density modifying agent with target cells to reach saturation, increasing the amount of antibody bound to density modifying agent to increase local concentration, or using a binding molecule with increased affinity for the cell surface target. In yet another example, some cells may display multiple cell surface markers and may be tagged (intentionally or not) with lower and higher density modifying agents. This can result in multiple populations of complexes comprising different cells and beads in different ratios. In some cases this can make separation between different cell populations difficult. This problem, if it arises, can be at least partially addressed, for example, by increasing the time of incubation of density modifying agent with target cells to reach saturation, increasing the amount of antibody bound to density modifying agent to increase local concentration, or by using a binding molecule with increased affinity for the cell surface target. In another exemplary approach, a subset of cells with multiple surface markers may be tagged with two different density modifying agents, such that the density change is modulated between no tag and all-of-one tag. It is understood that the above-discussed approaches may be used in various combinations of two or more approaches.

Methods of Cell Separation

Described in the present disclosure and included among the embodiments of the present invention are improved methods (processes) of cell separation by magnetic levitation. Such methods may also be referred to as “cell separation methods,” “methods of separating cells,” “methods of cell isolation,” “cell isolation methods,” “methods of isolating cells,” “cell concentration methods,” “methods of concentrating cells,” “cell segregation methods,” “methods of segregating cells,” and by other related terms and expressions, which are not intended to be limiting.

The methods described in the present disclosure are useful for separating one or more types of cells from a population of cells including multiple cell types. Such multiple cell types may include animal cells, including human cells and non-human animal cells, mixtures of human and non-human cells, plant cells, as well as cells of other origins, including, but not limited to, bacterial cells, protozoan cells, algal cells, etc. Multiple cell types may include dead cells, living cells, healthy cells, pathological cells, infected cells, transfected cells, or genetically modified cells. Cells separated according to the methods of the present disclosure may include cells in various states (for example, stem cells, differentiated cells, etc.). Cells separated according to the methods of the present disclosure can be directly obtained from an organism (or be an organism itself), or propagated or cultured. Cells can be subject to various treatments, storage or processing procedures before being separated according to the methods described in the present disclosure. Generally, the terms “cell,” “cells,” “cell type” (or the related terms and expressions) are not intended to be limited by their source, origin, manner of procurement, treatment, processing, storage or analysis, or any modification. Some non-limiting examples of the cell types that may be suitable for being separated by the methods described in the present disclosure are macrophages, alveolar type II (ATII) cells, stem cells, adipocytes, cardiomyocytes, embryonic cells, tumor cells, lymphocytes, red blood cells (erythrocytes), epithelial cells, ova (egg cells), sperm cells, T cells, B cells, myeloid cells, immune cells, hepatocytes, endothelial cells, stromal cells, and bacterial cells. A population of cells that includes multiple cell types may be derived from various types of samples, which are discussed elsewhere in the present disclosure.

Cell separation methods according to some of the embodiments of the present invention involve performing binding of density-modifying microparticles to a cell of a particular type or types (which can be referred to as “target cell” or “target cell type”) found in a population of cells comprising multiple cell types. Such binding can also be described as “forming a complex,” “complex formation” or “complexing.” Preferential binding of density-modifying microparticles to a target cell is accomplished by using binding molecules capable of specifically or selectively binding the target cell. Such molecules can be called “specific binding molecules.” An example of a specific binding molecule is an antibody specific against a cell surface marker or a molecule specific for a target cell type. Some examples of surface markers are CD45, CD3, CD4, CD8, CD19, CD40, CD56, CD11b, CD14, CD15, EpCAM, ICAM, CD235, HER-2, HER-3, CD66e, Integrins, E- P- L-Selectins, EGFR, EGFRVIII, PDGFR β, c-MET, MUC-1, OX-40, CD28, CD133, CD30 TNFRSF8, CTLA4, CD71, CD16α VCAM-1, Nucleolin, and Myelin Basic Protein. It is to be understood that different cells may have different surface markers. For example, human and non-human animal cells, such as mouse cells, may have different surface markers. Embodiments of the cell separation methods of the present invention may utilize more than one (one or more, two or more, three or more, four or more, etc.) specific binding molecule. In other words, embodiments of the cell separation methods of the present invention may utilize multiple specific binding molecules capable of forming complexes with different target cell types in a population of cells containing multiple cell types.

In the embodiments of the cell separation methods that involve binding of density-modifying microparticles to a cell of a particular type or types, the binding can be accomplished by various steps. For example, the binding can be accomplished by contacting, combining or incubating a density-modifying microparticle comprising a specific binding molecule with a population of cells comprising multiple cell types, potentially including a target cell type, under conditions in which the density-modifying microparticle binds individual cells of the target cell type to form complexes, each complex one or more microparticles bound to a cell of the target cell type. In another example, the binding can be accomplished by contacting, combining or incubating a specific binding molecule with a population of cells comprising multiple cell types, potentially including a target cell type, However, embodiments of the cell separation methods according to the present invention need not include any steps related to binding of density-modifying microparticles to cells. Complexes of density-modifying microparticles and target cells can be formed before the start of the method and be provided at the beginning of the cell separation methods according to the embodiments of the present invention. In other words, the method can start with a step of providing a complex of one or more density-modifying microparticles and a cell of a target cell type (or target cell), optionally included in a population of cells comprising multiple cell types.

Some embodiments of cell separation methods according to the present invention include a step or steps related to forming a suspension, in a paramagnetic fluid medium, of a complex of one or more density-modifying microparticles and a cell of a target cell type, and a plurality of the cells of the multiple cell types. In some embodiments, such a suspension may be provided at the start of the method. Cell separation methods according to the embodiments of the present invention involve introducing the suspension into a processing channel of a flowcell cartridge of a magnetic levitation system. The flowcell cartridge comprises at least one outlet channel, and a processing channel having a length and a vertical height. Cell separation methods according to the embodiments of the present invention involve exposing the processing channel to a magnetic field for a period of time sufficient for at least some of the complexes (or at least one complex) of one or more density-modifying microparticles and a cell of a target cell type to separate from the cells of the multiple cell types not bound to density-modifying microparticles, thereby forming a first portion of the suspension enriched with the complex relative to the suspension, and a second portion of the suspension depleted by the complex relative to the suspension. The exposure to the magnetic field can be performed in a stop-flow mode or continuous flow mode of the flowcell cartridge.

In some embodiments of the cell separation methods, a vertical position of the flowcell cartridge in the magnetic field is changeable, which may affect levitation height of the complexes (or at least one complex) of one or more density-modifying microparticles and a cell of a target cell type relative to magnets of the magnetic levitation system. Changing a vertical position of the flowcell cartridge may be advantageously used to access different portions of the suspension. In some embodiments of the cell separation methods, the composition of the paramagnetic fluid can be adjusted to improve cell separation. For example, the concentration of the paramagnetic compound changes the physical space occupied by a range of densities, such that it is possible to target a specific range of densities within the separation channel by adjusting the concentration of the paramagnetic compound. With the increase in the concentration of the paramagnetic compound in the paramagnetic fluid, the range of the particle densities that can be levitated between the magnets becomes broader. However, if the magnet spacing is not adjusted in this scenario, the physical separation distance between the particles of different densities becomes smaller. Conversely, with the decrease of the concentration of the paramagnetic compound in the paramagnetic fluid, the range of the particle densities that can be levitated between the magnets narrows, but the physical separations distance between the particles of different densities increases. Accordingly, in the methods of cells separation according to the embodiments of the present invention, the concentration of the paramagnetic compound in the paramagnetic fluid and/or the magnet spacing in the magnetic levitation system may be adjusted to optimize purity and/or yield of the separation product (particle of interest).

In the embodiments of the cell separation methods that utilize multiple specific binding molecules capable of forming complexes with different target cell types in a population of cells containing multiple cell types, more than two portions of the suspension may be formed after exposure to the magnetic field. For example, if a method utilizes two different specific binding molecules capable of binding to two different target cell types in a population of cells containing multiple cell types, two different types of complexes may be formed in a suspension upon exposure to the magnetic field: a first complex of first type of microparticles and individual cells of a first target type, and a second complex of second type of microparticles and individual cells of a second target type. In such a situation, the first type of microparticles and the second type of microparticles are selected such that the density of the first complex is different from the density of the second complex, and is also different from the density of the other types of cells in the population. In one example, at least three different portions of the suspension will then form in a processing channel of a flowcell cartridge upon its exposure to the magnetic field: a portion enriched by the first complex, a portion enriched by the second complex, and a portion (or portions) depleted of the first complex and the second complex. In this situation, different portions of the suspension (which can be referred to as “fractions”) may require same or different periods of exposure to the magnetic field to form.

Cell separation methods according to the embodiments of the present invention may further include withdrawing different portions of the suspensions from the processing channel of the flowcell cartridge or from the flowcell cartridge altogether. Withdrawing of different portions or fractions can be performed through one or more outlet channels of the flowcell cartridge, and may involve flowing the suspension along the length of the processing channel.

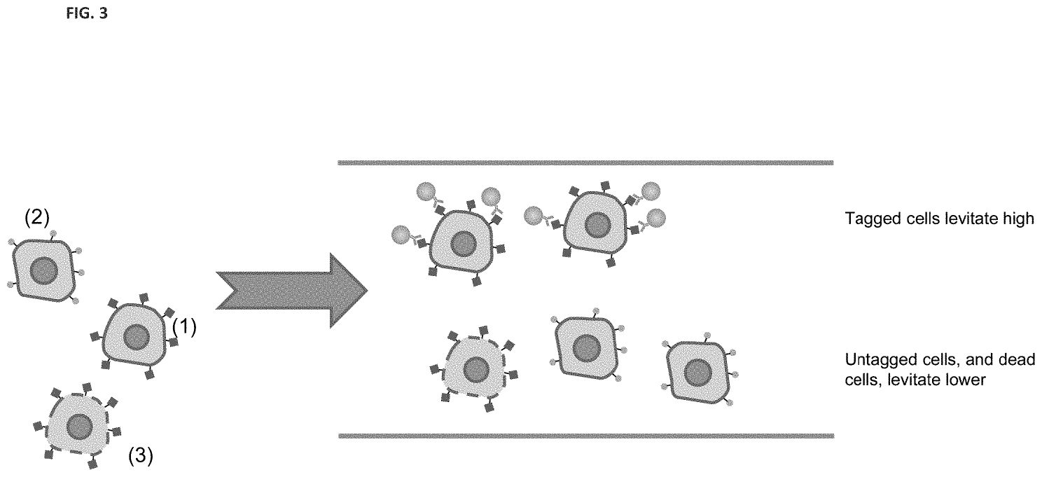

An exemplary embodiment of a cell separation method is schematically illustrated in FIG. 3. An embodiment illustrated in FIG. 3 is an example of a method of cell separation that uses microparticles with buoyant density lower than that a target cell type (first type) in a population of cells containing at least two cell types, a first type and a second type, and also including some dead cells. Live cells of two different cell types ((1)—live cells of the first type; ((2)—live cells of the second type) have different surface markers (such as proteins, carbohydrates or other biological molecules). The population of cells is contacted with non-magnetic microparticles coupled to antibodies capable of specifically binding to a surface marker of the first type of the two cell types. The non-magnetic microparticles bind to the surface marker of the first cell type, forming complexes with the cells of the first type. The density of the complexes is lower than the density of the non-complexed cells of the first type or the second type. When subjected to magnetic levitation, the complexes levitate higher in the processing channel of the flowcell cartridge than the cells of the second type, which did not form the complexes with the microparticles. Dead cells of the first type (3) also levitate lower than the complexes. The dead cells of the second type may not have as much of the surface marker exposed, or may have lower density than live cells of the first type. The fraction enriched in the complexes formed by the live cells of the first type is withdrawn from the flowcell cartridge, resulting in isolation of live cells of the first type.

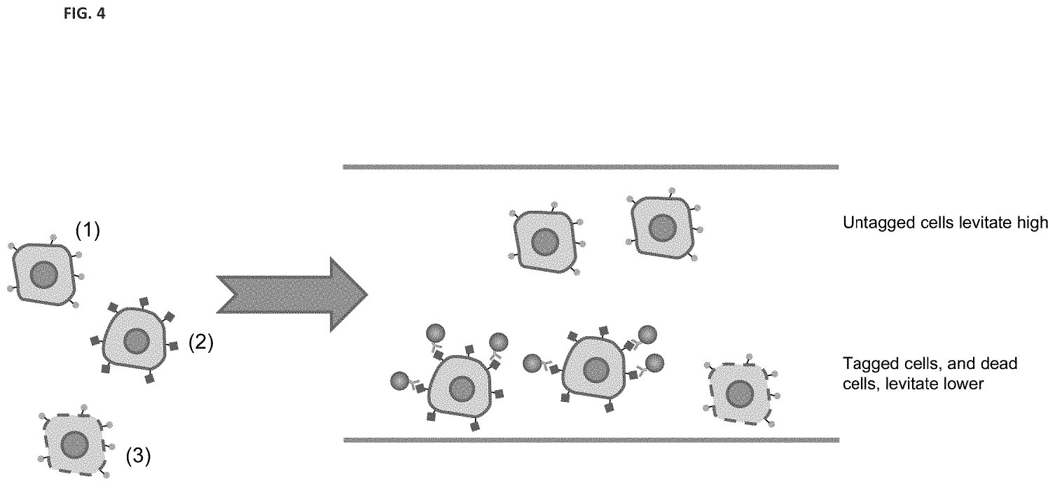

Another exemplary embodiment of a cell separation method is schematically illustrated in FIG. 4. An embodiment illustrated in FIG. 4 is an example of a method of cell separation that uses microparticles with buoyant density higher than that a target cell type (second type) to separate a target cell type from a population of cells containing at least two cell types, a first type (1) and a second type (2), and also including some dead cells of the first type (3). Live cells of two different cell types have different surface markers (such as proteins, carbohydrates or other biological molecules). The population of cells is contacted with the microparticles coupled to antibodies capable of specifically binding to a surface marker of the second type of the two cell types. The microparticles bind to the surface marker of the second cell type, forming complexes with the cells of the second type. The density of the complexes is higher than the density of the non-complexed cells of the first type or the second type. When subjected to magnetic levitation, the complexes levitate lower in the processing channel of the flowcell cartridge than the cells of the first type, which did not form the complexes with the microparticles. Dead cells also levitate lower than the non-complexed cells. The fraction depleted in the complexes is withdrawn from the flowcell cartridge, resulting in isolation of live cells of the first type.

### Systems and Kits for Particle Separation

Described in the present disclosure and included among the embodiments of the present invention are kits and systems useful for separation of particles, such as cells, by magnetic levitation. An exemplary kit comprises one or more types of density-modifying non-magnetic microparticles capable of forming complexes with individual cells. Such non-magnetic microparticles are described elsewhere in the present disclosure. In addition to one or more types of density-modifying non-magnetic microparticles, the kit can include a paramagnetic fluid medium. In addition to one or more types of density-modifying non-magnetic microparticles, the kit may include one or more linking agents. The kit may also include one or more of the other components, such as, but not limited to, antibodies, conjugating agents, buffers (including, but not limited to, buffers formulated to decrease non-specific binding of particles to non-target cells), flow cell cartridges, or materials designed to optimize depletion or recovery of target cells from a mixed cell population

An exemplary system for separation of particles is a system of cell separation, which includes one or more types of density-modifying microparticles that are capable, alone or in combination with other reagents, of preferentially binding to cells of a target cell type in a population of cells comprising multiple cell types and forming complexes of the microparticle and a cell of the target cell type. The system further includes a flowcell cartridge described elsewhere in the present disclosure, a station comprising a holding block for the flowcell cartridge, and one or more magnets positioned to expose the processing channel of the flowcell cartridge located in the holding block to a magnetic field.

### EXAMPLES

The following examples are offered to illustrate, but not to limit the claimed invention.

### Example 1: Separation of Jurkat Cells Using Microbubbles

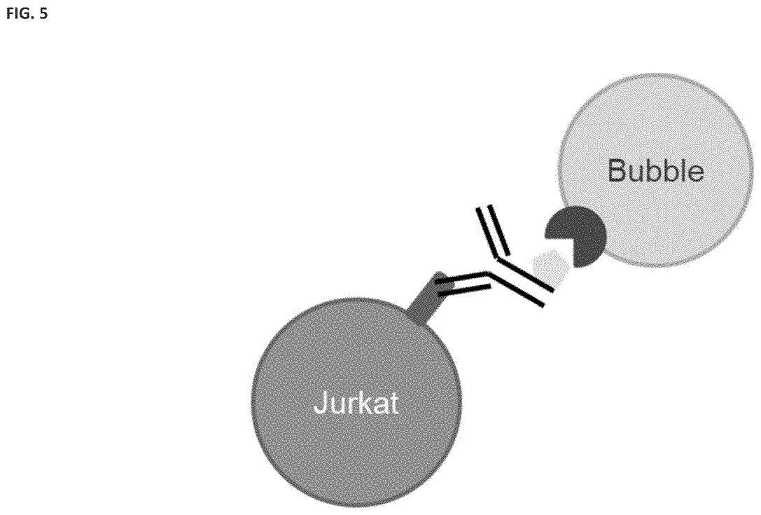

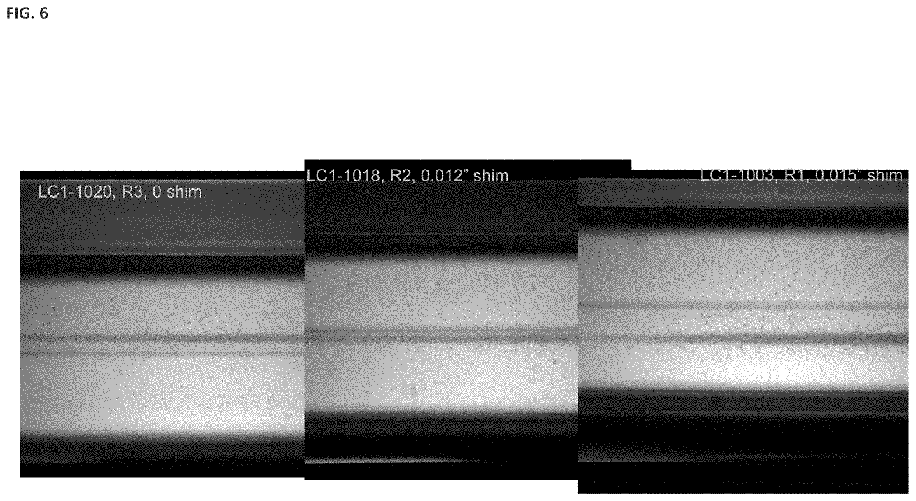

Separation of Jurkat cells (“Jurkat”) using microbubbles was accomplished and is illustrated in FIG. 5-FIG. 7. SIMB3-4 microbubbles were purchased from Advanced Microbubbles Inc. (Newark, Calif.). Jurkat cells of a human T-cell line (ATCC TIB-152, Jurkat clone E6.1) were stained with Calcein AM (Thermo Fisher Scientific Inc.) at 10 μM final concentration to provide green fluorescence and washed 3 times to remove excess stain. Stained Jurkat cells were mixed with H358 cells (NCI-H358 obtained from Berkeley Cell Culture Facility, Berkeley, Calif.), a human lung cell carcinoma cell line that does not express CD45, resulting in a sample containing about 50% H358 cells and about 50% Jurkat cells (“H358/Jurkat mixture”). The H358/Jurkat mixture was incubated with biotinylated anti-CD45 antibody at 1.25 μg/ml (clone HI30, Biolegend, San Diego, Calif.) for 1 hour on ice, and then washed 4 times to remove the unbound antibody. Stained cells were mixed with streptavidin-conjugated microbubbles at a microbubble:cell ratio of about 20:1 for 15 minutes at room temperature with mixing at 500 rpm in a Thermomixer® (Eppendorf, Enfield, Conn., USA). Levitation agent containing 1 M Gadobutrol was then added to the cell/microbubble mixture to a final concentration of 75 mM and the resulting levitation suspension was loaded into a flowcell cartridge of LeviCell™ magnetic levitation platform (Levitas Bio, Menlo Park, Calif., USA). FIG. 5 schematically illustrates the complex of biotinylated antibody-stained Jurkat cells bound to streptavidin-conjugated bubbles. The levitation suspension was exposed to magnetic field (“equilibrated”) for 20 min inside the LeviCell instrument, and then flowed through the flowcell. In a series of levitation experiments, the cartridges were either not raised off of the bottom magnet (standard configuration) or were raised off of the bottom of the magnet with a 0.012″ or a 0.015″ shim of brass. This presence of the shim raised the position of the flowcell cartridge between the two magnets, allowing the cartridge to sit higher in the magnetic field, as illustrated in FIG. 6. The above experiment illustrated the following embodiment of the present invention. The cells levitate in a processing channel of a flow cell cartridge at a fixed position in the magnetic field (that is, between the between the two magnets). The intrinsic position of a cell in magnetic field, which is influenced by the cell density and magnetic susceptibility, can be termed a “levitation profile.” By moving the flowcell up or down, the cells, while staying at their levitation profile, can be positioned lower or higher in the flowcell, respectively. The ability to adjust the position of the levitated cell in the processing channel of the flowcell cartridge (“levitation height”) can be used to improve collection of the separated fraction.

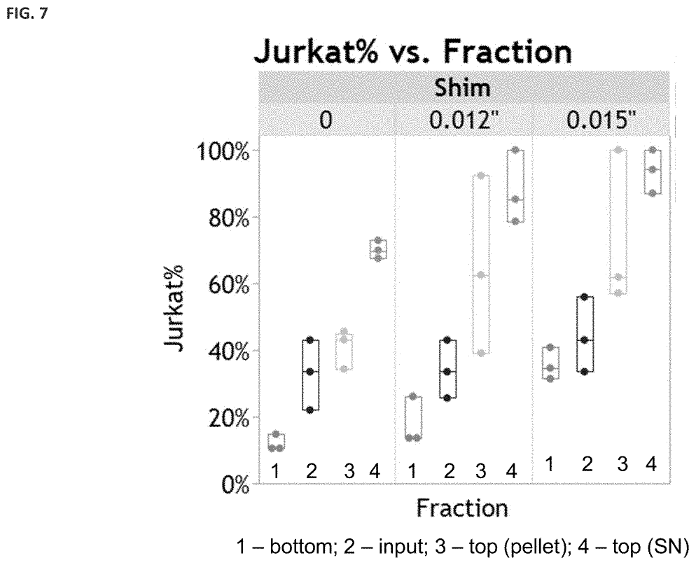

FIG. 6 shows photographic images of the cell mixture at the end of the equilibration phase in the flowcell using different shims, as indicated in the images. The darker band of cells near the middle of the flowcell corresponds to the H358 cells that have no microbubbles bound because they have no CD45 antibody bound to their surface. Jurkat cells bound to the microbubbles are levitating above the band of cells, but in this case did not form a single band. Depending on the cell size and number of the microbubbles bound to each cell, Jurkat-microbubble complexes levitated at a range of distances above their original position with some Jurkat cells levitating up to the top of the flowcell. When the flowcell was raised using the 0.015″ shim, the complexes that were at a height that was not visible prior to the raising of the flowcell (i.e were immobilized on the top of the channel) do become visible. This demonstrated that the Jurkat/bubble complexes were capable of raising the levitation height of Jurkat cells higher vs cells not bound with bubbles, with some complexes being raised as far up as the top of the flowcell. FIG. 7 shows the bar graphs summarizing the percentage of Jurkat cells present in the sample at the input, top, and bottom fractions (as indicated) of the flow cell cartridge. The cells were counted on single use hemocytometer chips using an Echo fluorescent microscope. Total cell counts from 4 corner grids were then divided by 4, then by 10,000 (the conversion factor for the hemocytometer chip) to get the cells/ml. The green fluorescent Jurkat cells were counted (due to the Calcein fluorescent stain) and compared to the total number of cells to get the % Jurkat value. The top fraction was withdrawn from the flowcell, centrifuged to pellet unbound cells and the supernatant was counted separately from the pellet to identify how many Jurkat cells were complexed with the microbubbles and thus floating in the supernatant. The results showed that the supernatant of the top fraction was enriched by the Jurkat cells, as compared to the input levitation sample, and that the purity of Jurkat cells increased when shims were used to raise up the flowcell. The purity of Jurkat cells present in the pelleted cells from the top fraction also increased as the flowcell was raised up with the shims. The bottom fraction was depleted in Jurkat cells compared to the input levitation sample.

### Example 2: Separation of Jurkat Cells Using Polymeric Microbeads

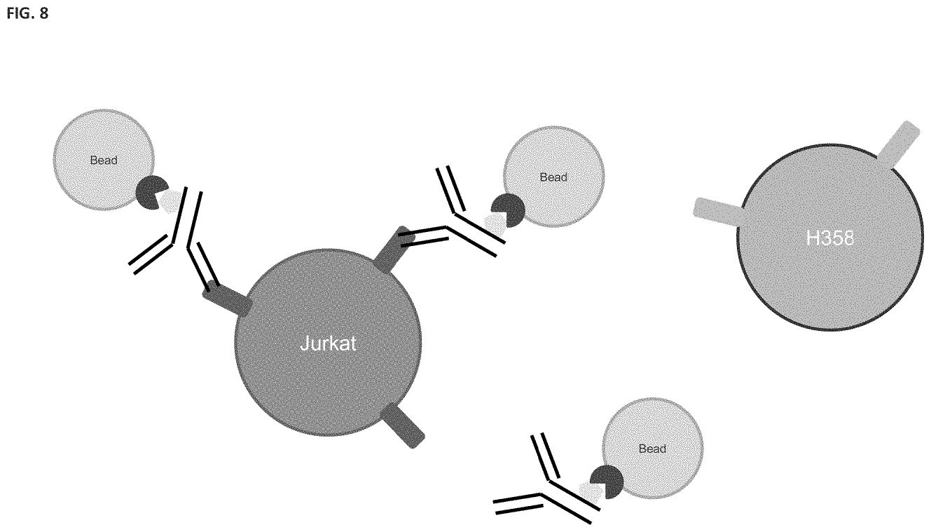

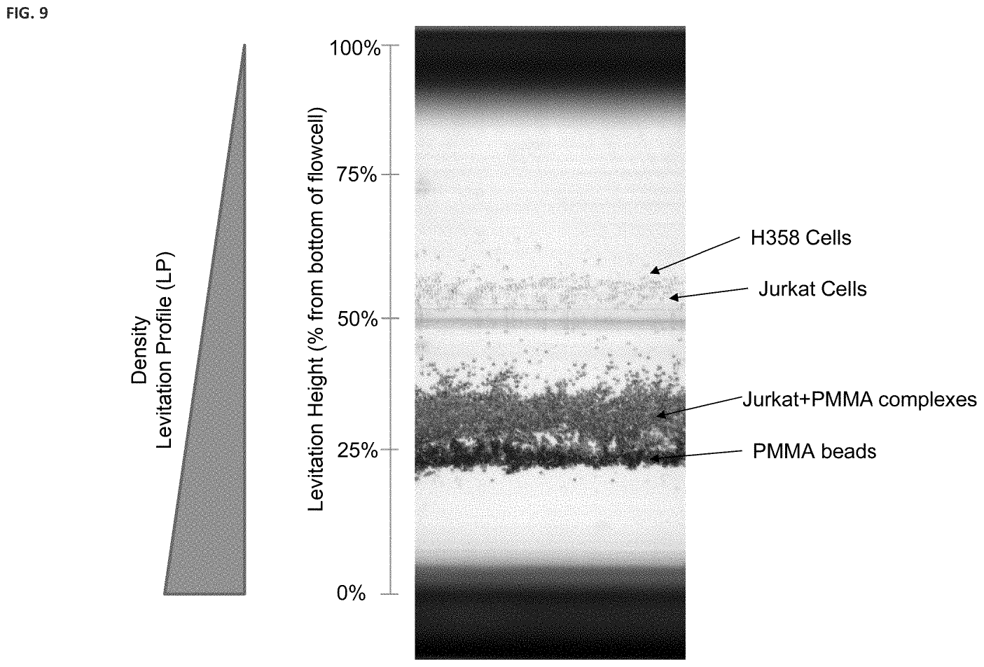

Separation of Jurkat cells using polymeric microbeads was accomplished and is illustrated in FIG. 8 and FIG. 9. 12 μm DiagPoly™ Streptavidin PMMA beads (“microbeads”) were sourced from Creative Diagnostics (Alpharetta, Ga.). Jurkat cells (human T-cell line ATCC TIB-152, Jurkat clone E6.1) were stained with Calcein AM as described in Example 1 in order to provide green fluorescence. H358 cells (NCI-H358 obtained from Berkeley Cell Culture Facility, Berkeley, Calif.) were stained with CellTracker Red CMPTX (Thermo Fisher Scientific Inc.) at 10 μM final concentration to provide red fluorescence. Both types of stained cells were washed 3 times to remove excess fluorescence stain. Stained Jurkat and H358 cells were mixed to ^{˜}15% H358 cells/^{˜}85% Jurkat cells (“H358/Jurkat mixture”). The H358/Jurkat mixture was incubated with biotinylated anti-CD45 antibody for 1 hour on ice as described in Example 1, and subsequently washed 4 times to remove the unbound antibody. The antibody-stained cells were then mixed with the microbeads at a bead-to-cell ratio of 1:1 for 5 minutes at room temperature with 500 rpm mixing in a Thermomixer® (Eppendorf, Enfield, Conn., USA). Levitation agent containing 1M gadobutrol was then added to the cell/microbubble mixture to a final concentration of 75 mM. The resulting levitation suspension was loaded into a flowcell cartridge of LeviCell™ magnetic levitation platform (Levitas Bio, Menlo Park, Calif., USA). and then flowed through the flowcell. FIG. 8 schematically illustrates the complex of Jurkat cells (“Jurkat”) bound to streptavidin-conjugated microbeads using a biotinylated antibody linker.

FIG. 9 is a composite photographic image (reproduced in greyscale) of brightfield, red and green fluorescence from a frame at the end of the 20 min equilibration step. Unbound H358 cells remain in the top band in the flowcell, along with a few untagged Jurkat cells (live untagged cells have the lowest density in the mixture and the lowest levitation profile). Below them are the Jurkat cells complexed with the PMMA microbeads. Their levitation profile (LP) depends on how many beads are attached to each cell, with more beads making the cells levitate lower in the flowcell. At the bottom are unbound PMMA microbeads, which have the highest density and highest levitation profile. In the magnetic levitation experiment illustrated in FIG. 9, the fraction containing H358 cells withdrawn from the flowcells was enriched to 47%, corresponding to 92% depletion of Jurkat cells. Example 3: Separation of Jurkat Cells Using Gold Microbeads

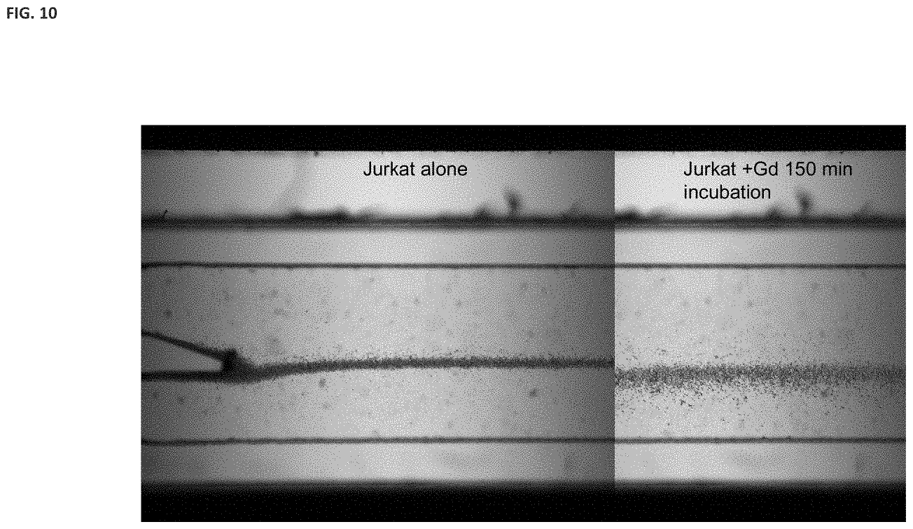

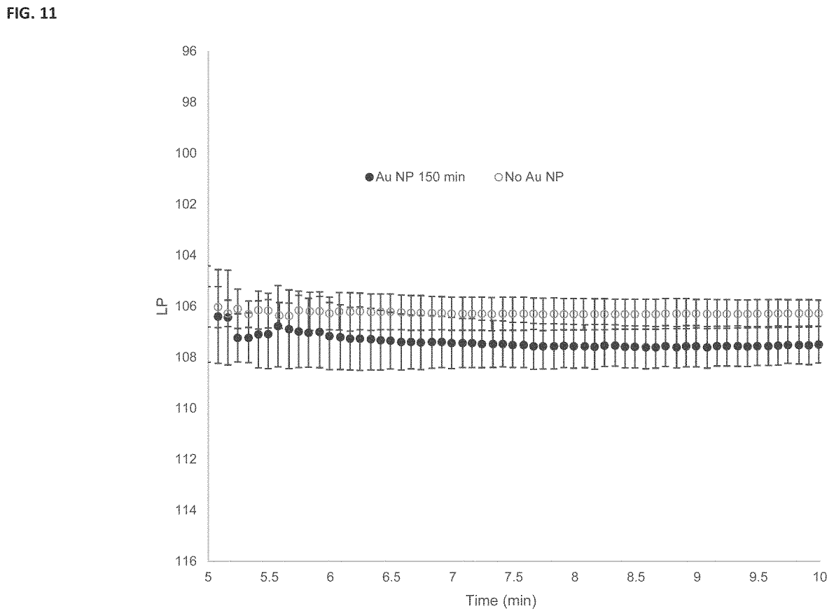

Separation of Jurkat cells using gold microbeads was accomplished and is illustrated in FIGS. 10 and 11. Jurkat cells (described in Example 1) were labeled with biotinylated anti-CD45 antibody at 50 μg/ml (clone HI30, Biolegend, San Diego, Calif.) for 1 hour on ice, and then washed 4 times to remove the unbound antibody. Thus labeled Jurkat cells were either incubated with gold nanoparticles for 150 minutes (“Jurkat+Gd 150 min incubation” in FIG. 10 and “Au NP 150 min” in FIG. 11). Unlabeled Jurkat cells not incubated with gold nanoparticles (“Jurkat alone” in FIG. 10 and “No Au NP” in FIG. 11) were used as a control. Magnetic levitation was performed substantially as described in the previous examples. The levitation profile (LP in FIG. 11) was calculated from the position in the flowcell where the middle of the band of cells levitated. Calculations of average levitation values were performed from images taken during the experiment illustrated in FIG. 10. The bars in FIG. 11 represent the thickness of the band of cells. When the levitation suspension reached equilibrium, after approximately ^{˜}10 minutes levitation, the cells incubated with gold nanoparticles for 150 minutes levitated with a higher LP, translating to levitating lower in the flowcell and thus having a higher density compared to cells without any gold nanoparticles.

### Example 4: Effect of the Microparticle Size on Cell Separation

It may be advantageous to use lower amount of density-modifying agent for cell separation. However, it needs to be ensured that each cell has at least one microparticle bound, which requires a higher microparticle-to-cell ratio to reach a saturating equilibrium binding concentration. Larger microparticles are supplied at the same weight/volume concentration (mass concentration) as smaller microparticles, so a given volume of 12 μm beads in suspension contains about 8 times fewer microparticles than the same volume of 5 μm microparticles. In addition, with the increase in microparticle size, its surface area also increases, thus requiring more linking agent per microparticle. Since the linking agent is immobilized to the surface of the bead, the increased amount of the linking agent per microparticle with the increase in microparticle size does not directly translate to increased “solution phase” linking agent concentration. Given any microparticle type of a certain density but differing in size, and at the same mass concentration, and assuming that the linking agent covers a fixed proportion of the microparticles surface area, independent of the microparticle size, the linking agent molecules are better distributed across a larger number smaller microparticles (at fewer linking agent molecules per microparticle) than across fewer larger microparticles. Larger microparticles also take up a large amount of space in the processing channel of the flowcell cartridge, effectively setting an upper limit to how many microparticles can be used. The following experiments were conducted to test the microparticle size on cell separation.

Separation of Jurkat cells using polymeric microbeads was accomplished substantially as described in Example 2, with following modification. Separate experiments were conducted with 12 μm and 5 μm streptavidin-conjugated PMMA beads (“microbeads”). The microbeads were coated with biotin-anti CD45 antibody to bind all available biotin binding sites. Biotin binding capacity for 5 μm microbeads was estimated by scaling down biotin binding capacity of 12 μm microbeads. Antibody-coupled microbeads were incubated with 15% CD45^{neg}/85% CD45^{pos} cell mixture at 1:1 and 60:1 bead-to-cell ratios for 12 μm and 5 μm microbeads, respectively. The 12 μm beads at a 1:1 bead-to-cell ratio depleted about 55% of CD45^{pos} cells. 12 μm microbeads bound to CD45^{pos} cells sufficiently reduced the levitation height of the resulting complexes, but the fraction was diffuse in the processing channel, and many microbeads were not bound to the cells. 5 μm microbeads depleted about 85-87% of CD45^{pos} cells, but required a bead-to-cell ratio of 60:1. Higher bead-to-cell ratios would likely have improved the performance of the 12 μm beads, however a 60:1 ratio with the same concentration of antibody to coat the beads would have required almost 12 times as much antibody as the 5 μm beads. Additionally, that high a number of large beads would have far surpassed the capacity of the flowcell processing channel.

It was theoretically predicted and experimentally confirmed that the most effective, binding of the levitation-height altering agent or magnetic agent to cells was performed when using the microparticles of smallest size possible, such that the product of the number of the linking agent molecules per microparticle and the number of the microparticles per unit solution volume is as close as possible to the saturating concentration of that particular linking agent in solution. For example, two different types of microparticles made from the same material and having the same density can have different sizes, the first microparticle 5 μm in diameter, and the second microparticle 1 μm in diameter. The 5 μm microparticle has 125× the particle volume and 25× the surface area compared to the 1 μm microparticle. Assuming that a fixed surface area required for every antibody binding event, the 5 μm microparticle can bind 25× linking agent molecules than the 1 μm microparticle. However, in solution, at the same weight-per-volume concentration of microparticles, the 1 μm microparticle will be 125× more concentrated. Thus, the effective solution concentration of the microparticle-bound linking agent is 5× higher for the 1 μm microparticles than for the 5 μm microparticles, with the same weight-per-volume concentration. Thus, it is easier to get to saturating concentration of microparticle-bound linking agent in solution by using smaller microparticles and using a higher number of microparticles per unit volume. It was experimentally determined that, to achieve effective cell separation on LeviCell™ magnetic levitation platform, the size of the microparticles has to be small enough to reach saturating concentration in solution, and large enough to effectively alter the density of the complex of the cell with the density-modifying agent. The effect of the microparticle size on the density of the complex of the cell with the density-modifying agent is illustrated in FIG. 13, which is discussed elsewhere in the present disclosure.

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

What is claimed is:

1. A method of cell separation, comprising:
a) binding a first density modifying agent to a cell of a first type in a population of cells comprising multiple cell types, wherein the first density modifying agent comprises a first nonmagnetic microparticle and a first linking agent that preferentially binds to cells of the first type, thereby forming a first complex, said first complex comprising the first density modifying agent bound to an individual cell of the first type;
b) forming a suspension in a paramagnetic fluid medium, the suspension comprising a plurality of the first complexes and a plurality of the cells of the multiple cell types;
c) introducing the suspension into a processing channel of a flowcell cartridge; and,
d) exposing the processing channel to a magnetic field for a first period of time sufficient for at least some of the first complexes to separate in the processing channel from the cells of the multiple cell types not bound by the first density modifying agent, thereby forming a first portion of the suspension, wherein the first portion is enriched with the first complex relative to the suspension, and a second portion of the suspension, the second portion depleted in the first complex relative to the suspension.

2. The method of claim 1, wherein the first linking agent comprises a first antibody or a domain of the first antibody capable of specifically binding to a first moiety on a surface of the cell of the first type.

3. The method of claim 2, wherein the binding of the first density modifying agent comprises:
binding of the first antibody or a domain of the first antibody capable of specifically binding to the first moiety on a surface of the cell of the first type; and,
binding of the first antibody or the domain of the first antibody to the first non-magnetic microparticle.

4. The method of claim 1, wherein the first linking agent binds to the first non-magnetic microparticle by first one or more covalent or non-covalent interactions.

5. The method of claim 1, further comprising:
e) withdrawing at least part of the first portion or withdrawing at least part of the second portion from the processing channel.

6. The method of claim 5, further comprising:
f) withdrawing at least part of the first portion or withdrawing at least part of the second portion from the flowcell cartridge.

7. The method of claim 1, further comprising:
in step (a), binding a second density modifying agent to a cell of a second type in the population of cells comprising multiple cell types, wherein the second density modifying agent comprises a second non-magnetic microparticle and a second linking agent that preferentially binds to cells of the second type of the multiple cell types,
wherein the binding of the second density modifying agent is under conditions in which the second density modifying agent binds individual cells of the second type to form second complexes, each second complex comprising the second density modifying agent bound to a cell of the second type,
wherein density of the second complex is not the same as any of the density of the cell of the second type, the density of the first complex, and the density of the other types of cells of the multiple cell types;
in step (b), the suspension further comprising a plurality of the second complexes;
in step (d), exposing the flowcell cartridge to the magnetic field for a second period of time sufficient for at least some of the second complexes to separate in the processing channel from the cells of the multiple cell types not bound by the second density modifying agent, from the first complex, and from the other types of cells of the multiple cell types, thereby forming a third portion of the suspension, the portion enriched with the second complex relative to the suspension, and a fourth portion of the suspension, the fourth portion depleted by the second complex relative to the suspension.

8. The method of claim 7, wherein the second linking agent comprises a second antibody or a domain of the second antibody capable of specifically binding to a second moiety on a surface of the cell of the second type.

9. The method of claim 4, wherein the one or more non-covalent interactions of the first lining agent or the second linking agent comprise, independently, one or more of an interaction of biotin with an avidin-like molecule, an antibody-antigen interaction, or an interaction between an antibody-binding protein or a domain of the antibody-binding protein and its binding partner.

10. The method of claim 1, wherein step (d) is performed in a stop-flow mode or in a continuous flow mode of the flowcell cartridge.

11. The method of claim 1, wherein a vertical position of the flowcell cartridge in the magnetic field is changeable.

12. The method of claim 1, wherein the first microparticle and/or the second microparticle is a polymeric microparticle.

13. The method of claim 12, wherein the polymeric microparticle is a polymeric microbead or a polymeric microbubble.

14. The method of claim 1 wherein the first microparticle is a glass microparticle.

15. The method of claim 14, wherein the glass microparticle is a glass microbead or a glass microbubble.

16. The method of claim 1 wherein the first microparticle is a non-magnetic metal microparticle.

17. The method of claim 16, wherein the non-magnetic metal microparticle comprises gold, silver, or platinum.

18. The method of claim 1, wherein the one or more cells of the first type and/or the one or more cells of the second type are live cells.

19. A magnetic levitation kit comprising a paramagnetic fluid medium and one or more of density modifying agents or separate components of the one or more density modifying agents, capable of forming complexes with individual cells, wherein density of each of the complexes is different than density of the individual cells, and wherein each density modifying agent comprises a non-magnetic microparticle and a linking agent that preferentially binds to a target cell type.

20. A system for cell separation, comprising
a first non-magnetic microparticle capable, alone or in combination with first other reagents, of preferentially binding to cells of a first type in a population of cells comprising multiple cell types and forming a first complex of the microparticle and a cell of the first type, the first complex having density that is different from density of the cell of the first type and from other types of cells of the multiple cell types;
a flowcell cartridge comprising a first outlet channel and a processing channel;
a station comprising a holding block for the flowcell cartridge and one or more magnets positioned to expose the processing channel of the flowcell cartridge located in the holding block to a magnetic field,
wherein exposing to the magnetic field the processing channel of the flowcell cartridge containing a suspension of the cells of the multiple cell types in a paramagnetic fluid medium allows the first complex to separate in the processing channel from the cells of the multiple cell types not bound by the first non-magnetic microparticle and from the other types of cells of the multiple cell types.
